(19) United States
(12) Patent Application Publication
(10) Pub. No.: US 2025/0161264 A1
(43) Pub. Date: May 22, 2025

(54) (AMPAR-PAM)-NMDA RECEPTOR ANTAGONIST COMBINATION THERAPY FOR TREATMENT OF MENTAL CONDITIONS AND DISORDERS

(71) Applicant: Gilgamesh Pharmaceuticals, Inc., New York, NY (US)

(72) Inventors: Andrew Kruegel, Millington, NJ (US); Laszlo Kiss, Madison, CT (US)

(21) Appl. No.: 18/837,417

(22) PCT Filed: Feb. 10, 2023
(86) PCT No.: PCT/US2023/012790

§ 371 Date: Aug. 9, 2024

Related U.S. Application Data
(60) Provisional application No. 63/309,271, filed on Feb. 11, 2022.

(51) Int. Cl. A61K 31/381; A61K 31/135; A61K 45/06

(52) U.S. Cl. CPC A61K 31/381; A61K 31/135; A61K 45/06

(57) ABSTRACT

A pharmaceutical composition comprising the following components: (i) a positive allosteric modulator (PAM) of the AMPA receptor (e.g., an ampakine or biarylpropylsulfonamide); and (ii) an NMDA receptor antagonist, such as ketamine or an analogue thereof. Also described herein is a method for treating a mental disorder or condition (e.g., depression, anxiety, a substance use disorder, obsessive-compulsive and related disorders, or neurocognitive disorder), the method comprising administering to a subject having the mental disorder pharmaceutically effective amounts of components (i) and (ii), wherein components (i) and (ii) may be administered to the subject as a single pharmaceutical composition in which therapeutic compounds (i) and (ii) are present in pharmaceutically effective amounts, or components (i) and (ii) are administered to the subject in separate administrations within 48 or 24 hours of each other.

### CROSS REFERENCE TO RELATED APPLICATIONS

This application claims the benefit of U.S. Provisional Patent Application No. 63/309,271, filed Feb. 11, 2022, the contents of which is incorporated herein by reference in its entirety.

### FIELD OF THE INVENTION

The present invention generally relates to the treatment of a range of mental conditions and disorders by administering a positive allosteric modulator (PAM) of the AMPA receptor in combination with an NMDA receptor antagonist. The present invention more specifically relates to the treatment of depression (e.g., major depressive disorder), anxiety, bipolar disorder, mood disorders, obsessive-compulsive disorder, drug addiction, and neurocognitive disorders.

### BACKGROUND

Approximately one-third of patients with major depressive disorder (MDD) fail to achieve remission of their symptoms, even after multiple rounds of treatment with several known classes of antidepressants, including selective serotonin reuptake inhibitors (SSRIs) (Rush et al. N. Engl. J. Med., 354(12), 1231-1242, 2006). This high prevalence of treatment-resistant depression (TRD) makes clear the need for new, more efficacious pharmacotherapies for depression that will target new mechanisms and/or patient populations. In recent years, ketamine, a drug long used as a dissociative anesthetic, has attracted considerable attention for its secondary use as a rapid-acting antidepressant with robust efficacy, even in patients with TRD (Zarate et al., Arch. Gen. Psychiatry, 63(8), 856-864 2006; Berman et al., Biol. Psychiatry, 47(4), 351-354, 2000). The antidepressant effects of the drug are also notable in that they persist for days or weeks after a single administration. Importantly, the S enantiomer of ketamine (S-ket) has recently been approved by the United States Food and Drug Administration as a treatment for depression.

Unfortunately, the potent dissociative anesthetic effects of ketamine, S-ket, and other NMDA receptor antagonists make these drugs attractive to recreational drug users. In view of the significant potential for abuse of NMDA receptor antagonist drugs, such drugs are generally only available under limited and tightly controlled conditions, and under the supervision of a medical provider specializing in psychoactive treatments. Thus, there would be a significant benefit in a method of treating depression or other psychiatric disorder (e.g., anxiety, bipolar disorder, mood disorders, obsessive-compulsive disorder, drug addiction, and neurocognitive disorders) by administration of an NMDA receptor antagonist, but where the NMDA receptor antagonist is at a dose that does not induce a dissociative anesthetic effect in the patient while effectively treating the patient for the psychiatric disorder. Moreover, a method of treatment that diminishes the risk of abuse and overall danger in treatment using an NMDA receptor antagonist would also provide the advantage of no longer requiring such limited and controlled conditions. Lastly, a method of increasing the durability of the therapeutic effect resulting from administration of an NMDA receptor antagonist would allow the treatment to be administered less frequently, increasing convenience and decreasing risk.

### SUMMARY

In one aspect, the present disclosure is directed to a pharmaceutical composition that provides the positive treatment effects of an NMDA receptor antagonist for mental disorders but at lower doses, thereby diminishing the ability of the NMDA receptor antagonist to elicit a dissociative anesthetic effect, which in turn substantially diminishes the potential for abuse and also improves the safety of its use. Further, the invention increases the durability of the therapeutic effect resulting from administration of an NMDA receptor antagonist, thereby allowing the treatment to be administered less frequently, increasing convenience and decreasing risk for the patient. More specifically, the pharmaceutical composition includes or exclusively contains the following components: (i) a positive allosteric modulator (PAM) of the AMPA receptor (i.e., “AMPAR-PAM” compound); and (ii) an NMDA receptor antagonist compound. The pharmaceutical composition typically further includes: (iii) a pharmaceutically acceptable carrier.

In another aspect, the present disclosure is directed to a method of treating a mental disorder in a subject by administering a pharmaceutically effective amount of the AMPAR-PAM compound (therapeutic compound (i)) and NMDA receptor antagonist compound (therapeutic compound (ii)) to the subject in a combination therapy. The presence of the AMPAR-PAM compound has herein been unexpectedly found to favorably modulate the activity of the NMDA receptor antagonist by retaining and even synergistically enhancing its valuable effects in treating mental conditions. In some embodiments, the subject is administered a single pharmaceutical composition in which therapeutic compounds (i) and (ii) are present in pharmaceutically effective amounts for treating a mental disorder. In other embodiments, therapeutic compounds (i) and (ii) are administered to the subject in separate administrations within a sufficiently short timeframe for component (i) to effectively modulate the function of component (ii). For example, therapeutic compounds (i) and (ii) may be administered to the subject in separate administrations within 48 or 24 hours of each other. Notably, in some embodiments, component (i) and component (ii) are administered to the subject in amounts that are sub-therapeutic if administered alone, yet provide a synergistic effect that treats the mental disorder when administered in combination either as a single pharmaceutical composition or in separate administrations within a suitable time frame of each other.

### DETAILED DESCRIPTION

In a first aspect, the present disclosure is directed to a pharmaceutical composition that includes or exclusively contains the following components: (i) a positive allosteric modulator (PAM) of the AMPA receptor, also referred to herein as an “AMPAR-PAM compound”, and (ii) an NMDA receptor antagonist, also referred to herein as an “NMDAR-ant compound”. Typically, the pharmaceutical composition further includes a pharmaceutically acceptable carrier. Notably, component (i) may be referred to as “compound (i)” or “therapeutic compound (i)”, and similarly, component (ii) may be referred to as “compound (ii)” or “therapeutic compound (ii)”.

Component (i), the AMPAR-PAM compound, can be any of the AMPAR-PAM compounds known in the art. A host of AMPAR-PAM compounds are known, such as described in S. E. Ward et al., Br. J. Pharmacol., 160(2), 181-190, May 2010, the contents of which are herein incorporated by reference. As is well known, AMPAR-PAM compounds function as positive allosteric modulators (PAMs) of the ca-amino-3-hydroxy-5-methyl-4-isoxazole propionic acid (AMPA) receptor. The term “AMPA receptor” is herein used interchangeably with the term “AMPAR”. AMPARs are members of the class of ionotropic glutamate receptors, which are ligand-gated ion channels that become activated by glutamate, a neurotransmitter. These receptors are known to mediate much of the fast synaptic neurotransmission that occurs in the central nervous system.

In a first set of embodiments, the AMPAR-PAM compound is an ampakine. As is well known, some ampakines contain a benzamide base structure, often with the phenyl ring fused to another ring. An early example of such a compound is aniracetam. Some ampakines are identified as “CX” compounds, wherein the “CX” is followed by a set of numbers, e.g., “CX-516”.

Many of the CX ampakines have a structure within the following generic structure:

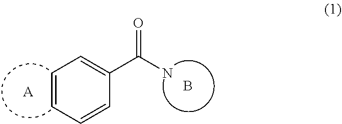

wherein ring A (shown fused to the phenyl ring, and optionally present) is typically a five- or six-membered carbocyclic or heterocyclic ring, and ring B is an N-containing ring typically containing four or five ring carbon atoms. Any one or more hydrogen atoms on rings A and/or B may independently be substituted with a hydrocarbon group, typically containing from 1-10 carbon atoms and optionally one or more heteroatoms selected from nitrogen, oxygen, and sulfur. In some cases, ring B is also fused to the phenyl ring to form at least a tricyclic ring system as follows:

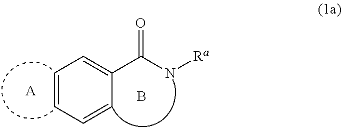

wherein R^{a} is typically a hydrocarbon group containing 1-6 carbon atoms and which may fuse with ring B to result in a tetracyclic ring system. Some examples of ampakines according to Formulas (1) and (1a) include CX-516 (Ampalex), CX-546, CX-554, CX-614, CX-691 (farampator), CX-717, CX-1632 (tulrampator), CX-1739, ORG-26576, ORG-24448, CX-701, CX-929, CX-1501, CX-1796, CX-1837, CX-1846, CX-1942, CX-2007, CX-2076, and S-70340. All pharmaceutically acceptable salts of any of the above ampakines, as well known in the art, may also be used.

In another set of embodiments, the AMPAR-PAM compound may alternatively have an arylpropylsulfonamide structure, or more particularly, a biarylpropylsulfonamide structure. Such AMPAR-PAM compounds may have a structure within any of the following generic structures:

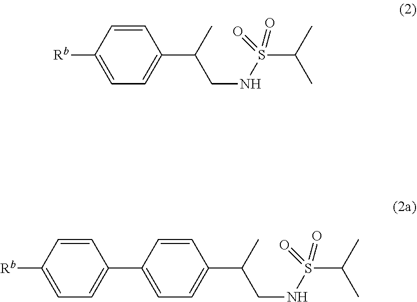

wherein R^{b} is typically a hydrocarbon group, typically containing from 1-12 carbon atoms and optionally one or more heteroatoms selected from nitrogen, oxygen, and sulfur. R^{b} may be or include, for example, a linear or branched alkyl or alkylene group, a cycloalkyl group, a heterocycloalkyl group, an aromatic group, or a heteroaromatic group. In some embodiments, R^{b} is a nitrile (CN) group. Moreover, one or both of the benzene rings shown in Formulas (2) or (2a), respectively, may be replaced with a heteroaryl group, such as a pyridine, pyrimidine, or thiophene ring. Some examples of arylpropylsulfonamide and biarylpropylsulfonamide compounds according to Formulas (2) and (2a) include LY-451395 (mibampator), LY-450108, LY-404187, LY-503430, PF-04958242 (BIIB-104; pesampator), LY-392098, LY-451646, PF-04778574, CMPDA, CMPDB, and (R,R)-PIMSD. In some embodiments, component (i) is or includes a biarylpropylsulfonamide, and the biarylpropylsulfonamide may be or include PF-04958242 (BIIB-104; pesampator). All pharmaceutically acceptable salts of any of the above arylpropylsulfonamides, as well known in the art, may also be used.

In another set of embodiments, the AMPAR-PAM compound may alternatively have a benzothiadiazide structure. The benzothiadiazide may be selected from, for example, BIIR-777, cyclothiazide, diazoxide, hydrochlorothiazide (HCTZ), IDRA-21, and S-18986.

In another set of embodiments, the AMPAR-PAM compound may alternatively have a racetam structure. The racetam may be selected from, for example, piracetam, aniracetam, phenylpiracetam, oxiracetam, pramiracetam, seletracetam, levetiracetam, coluracetam, fasoracetam, brivaracetam, methylphenylpiracetam, dimiracetam, nebracetam, rolziracetam, and nefiracetam. In some embodiments, the racetam is more specifically selected from piracetam, aniracetam, or combination thereof.

In another set of embodiments, the AMPAR-PAM compound may alternatively be selected from PEPA, PF-04701475, GVS-111, and TAK-653.

Component (ii), the NMDAR-ant compound, can be any such compound known in the art. A host of NMDAR-ant compounds are known, such as described in S. E. Tomek et al., Pharmaceuticals, 6(2), 251-268, 2013, the contents of which are herein incorporated by reference. As is well known, NMDA receptor antagonist compounds function to inhibit (i.e., antagonize) the N-methyl-D-aspartate receptor (NMDAR), which is a receptor for glutamate, a common excitatory neurotransmitter in the brain. By virtue of this ability, NMDAR-ant compounds are commonly used as anesthetics. NMDAR-ant compounds are also known to induce dissociative or hallucinogenic effects depending on the dosage.

In one set of embodiments, the NMDAR-ant compound is an arylcyclohexylamine. Many of the arylcyclohexylamines have a structure within the following generic structure:

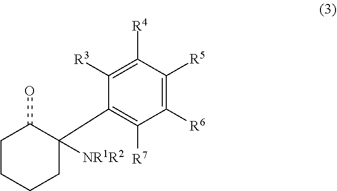

In Formula (3) above, R¹ and R² are independently selected from H and R^{a}, wherein R^{a} is independently selected from H, C₁₋₁₀ alkyl, C₂₋₁₀ alkenyl, C₂₋₁₀ alkynyl, and C₃₋₁₀ cycloalkyl, and wherein R^{a} optionally contains (i.e., optionally includes) one or more heteroatoms selected from O, N, S, and halogens, and wherein R¹ and R² are optionally interconnected to form an N-containing ring; and R³, R⁴, R⁵, R⁶, and R⁷ are independently selected from H, halogen, R^{b}, CF₃, —OR^{b}, —C(O)R^{b}, —C(O)O(R^{b}), —C(O)NR^{b}₂, —OC(O)R^{b}, —NR^{b}₂, —NR^{b}C(O)R^{b}, —NR^{b}C(O)NR^{b}—, —NO₂, —CN, —OP(═O)O₂R^{b}₂, —SR^{b}, —S(O)R^{b}, —SO₂R^{b}, and —SO₂NR^{b}₂, wherein R^{b} is independently selected from H, C₁₋₁₀ alkyl, C₂₋₁₀ alkenyl, C₂₋₁₀ alkynyl, C₃₋₇ cycloalkyl, aryl, and heteroaryl, any of which is optionally substituted with halogen atoms. The dashed double bond in Formula (3) indicates the optional presence of a keto group. The term “contains” or “includes”, in reference to the optional presence of one or more heteroatoms, refers to any manner in which the one or more heteroatoms may be present in any of the groups delineated under R^{a} or in any other groups disclosed elsewhere in this application. In one instance, the term “contains” or “includes” refers to substitution of one or two hydrogen atoms on a carbon atom by a heteroatom or a heteroatom-containing group (e.g., an oxygen atom replacing two hydrogen atoms on a carbon atom to result in a carbonyl group; or a fluorine atom, hydroxy group, amino group, or thiol group replacing a hydrogen atom). In another instance, the term “contains” or “includes” refers to an interruption of a carbon-carbon bond in a hydrocarbon group by a heteroatom or heteroatom-containing group (e.g., an oxygen atom interrupting an n-butyl group to result in a diethyl ether group, or an —NH— group interrupting an ethyl group to result in a —CH₂—NH—CH₃ group, or an oxygen atom interrupting a cyclobutyl group to result in a tetrahydrofuranyl group). In some embodiments, the alkyl, alkenyl, alkynyl, or cycloalkyl group does not contain a heteroatom. All pharmaceutically acceptable salts of any of the above arylcyclohexylamine compounds, as well known in the art, may also be used.

In some embodiments, the NMDAR-ant compound is an arylcyclohexylamine having the following generic structure:

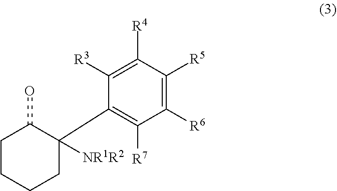

wherein, R¹ and R² are independently selected from H and R^{a}, wherein R^{a} is independently selected from H, C₁₋₅ alkyl, C₂₋₅ alkenyl, C₂₋₅ alkynyl, and C₃₋₅ cycloalkyl, and wherein R^{a} optionally contains (i.e., optionally includes) one or more heteroatoms selected from O, N, S, and halogens, and wherein R¹ and R² are optionally interconnected to form an N-containing ring; and R³, R⁴, R⁵, R⁶, and R⁷ are independently selected from H, halogen, R^{b}, CF₃, —OR^{b}, —C(O)R^{b}, —C(O)O(R^{b}), —C(O)NR^{b}₂, —OC(O)R^{b}, —NR^{b}₂, —NR^{b}C(O)R^{b}, —NR^{b}C(O)NR^{b}—, —NO₂, —CN, —SR^{b}, —S(O)R^{b}, —SO₂R^{b}, and —SO₂NR^{b}₂, wherein R^{b} is independently selected from H, C₁₋₄ alkyl, C₂₋₄ alkenyl, C₂₋₄ alkynyl, and C₃₋₅ cycloalkyl, any of which is optionally substituted with halogen atoms. All pharmaceutically acceptable salts of any of the above arylcyclohexylamine compounds, as well known in the art, may also be used.

In some embodiments, the NMDAR-ant compound is an arylcyclohexylamine having the following generic structure:

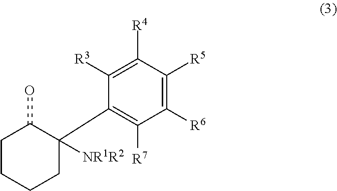

wherein, R¹ and R² are independently selected from H and R^{a}, wherein R^{a} is independently selected from H, C₁₋₃ alkyl, allyl, propargyl, and C₃₋₅ cycloalkyl, and wherein R^{a} optionally contains (i.e., optionally includes) one or more heteroatoms selected from O, N, S, and F, and wherein R¹ and R² are optionally interconnected to form a piperidine, pyrrolidine, or morpholine ring; and R³, R⁴, R⁵, R⁶, and R⁷ are independently selected from H, halogen, R^{b}, CF₃, —OR^{b}, —OC(O)R^{b}, —NR^{b}₂, —NO₂, —CN, wherein R^{b} is independently selected from H, C₁₋₄ alkyl, C₂₋₄ alkenyl, C₂₋₄ alkynyl, and C₃₋₅ cycloalkyl, any of which is optionally substituted with fluorine atoms. All pharmaceutically acceptable salts of any of the above arylcyclohexylamine compounds, as well known in the art, may also be used.

Some examples of arylcyclohexylamine compounds useful as component (ii) include ketamine, R-ketamine, S-ketamine, S-norketamine, N-ethylnorketamine, N-propylnorketamine, memantine, amantadine, phencyclidine (PCP), rolicyclidine (PCPy), PCM, PCPr, tenocyclidine (TCP), tiletamine, 2-oxo-PCP, 2-oxo-PCE, gacyclidine, 3-MeO—PCP, 3-OH—PCP, 4-MeO—PCP, deschloroketamine, 2-fluoro-deschloroketamine, eticyclidine (PCE), 3-MeO—PCE, 4-MeO—PCE, 3-OH—PCE, methoxetamine, methoxmetamine, methoxpropamine, methoxisopropamine, and hydroxetamine. In particular embodiments, component (ii) is or includes ketamine or an analogue thereof, such as any of the ketamine analogues provided above. In particular embodiments, component (ii) is selected from ketamine, R-ketamine, S-ketamine, and S-norketamine, or component (ii) is more particularly selected from ketamine, R-ketamine, and S-ketamine.

In some embodiments, component (ii) is selected from at least one of the following compounds:

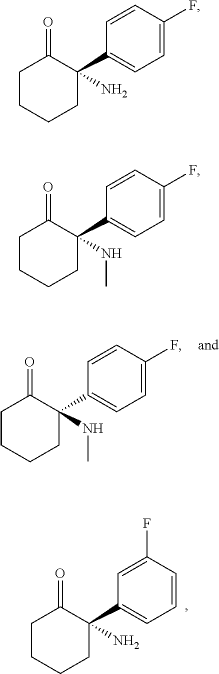 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]

or component (ii) is more particularly:

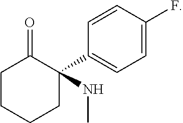 [SMILES: CN[C@@]1(c2ccc(F)cc2)CCCCC1=O]

In some embodiments, the NMDAR-ant compound is not an arylcyclohexylamine, but instead belongs to another molecular scaffold known to inhibit NMDAR. Examples of such compounds include 8A-PDHQ, dextromethorphan, dextromethorphan-OD3, dextrorphan, dextrallorphan, PD-137889, neramexane, memantine, NEFA, dizocilpine, lanicemine, diphenidine, ephenidine, isopropylphenidine, methoxphenidine, fluorolintane, xenon, krypton, nitrous oxide, etoxadrol, dexoxadrol, ibogaine, and noribogaine. In particular embodiments, the NMDAR-ant compound is selected from dextromethorphan and dextromethorphan-OD3.

In some embodiments, the NMDAR-ant compound selectively blocks or negatively modulates NMDA receptors containing the GluN2B (GRIN2B) subunit. Examples of such compounds include MIJ821, rislenemdaz, eliprodil, ifenprodil, besonprodil, traxoprodil, radiprodil, BMS-986169, BMS-986163, Ro 25-6981, Ro 04-5595, TCN 237, TCS 46b, and Co 101244.

In some embodiments, for any of the possibilities provided above for component (ii), the composition further includes a third compound (component iii), which is selected from quinidine and bupropion. In some embodiments, component (ii) is selected from dextromethorphan and dextromethorphan-OD3, and the composition further includes a third compound (component iii), which is selected from quinidine and bupropion.

In some embodiments, the NMDAR-ant compound is selected from:

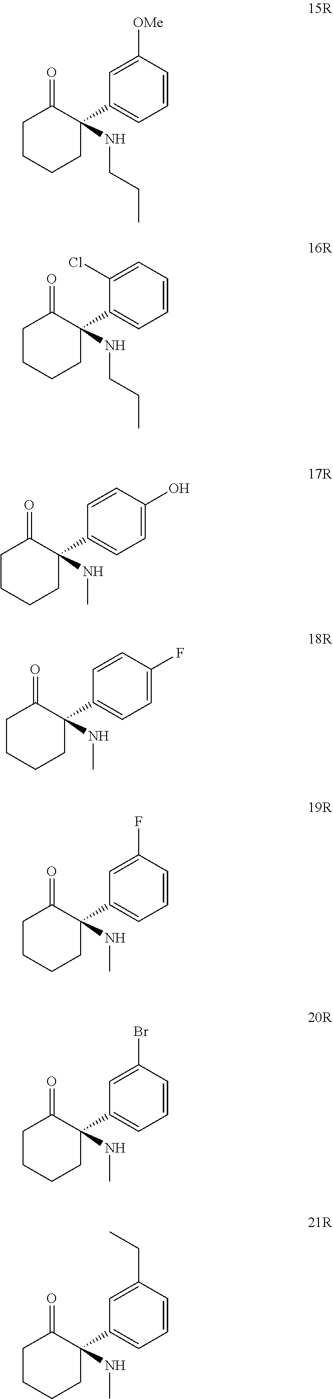

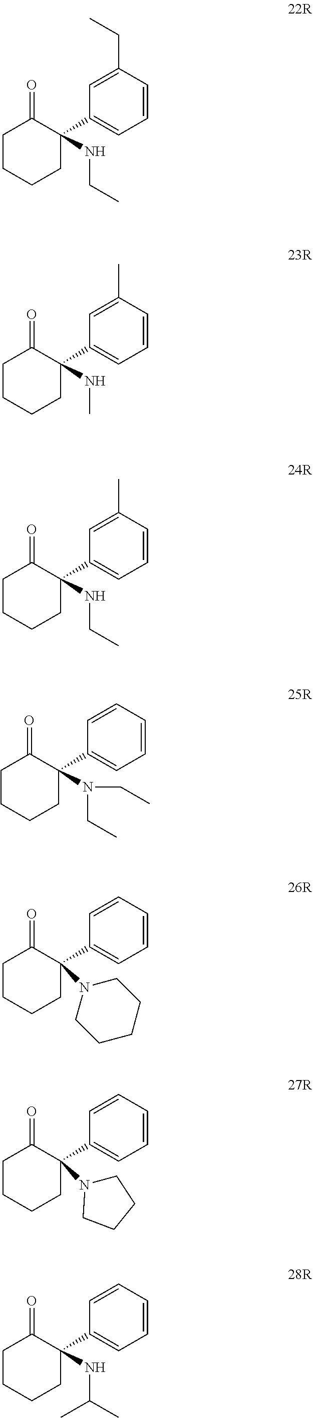

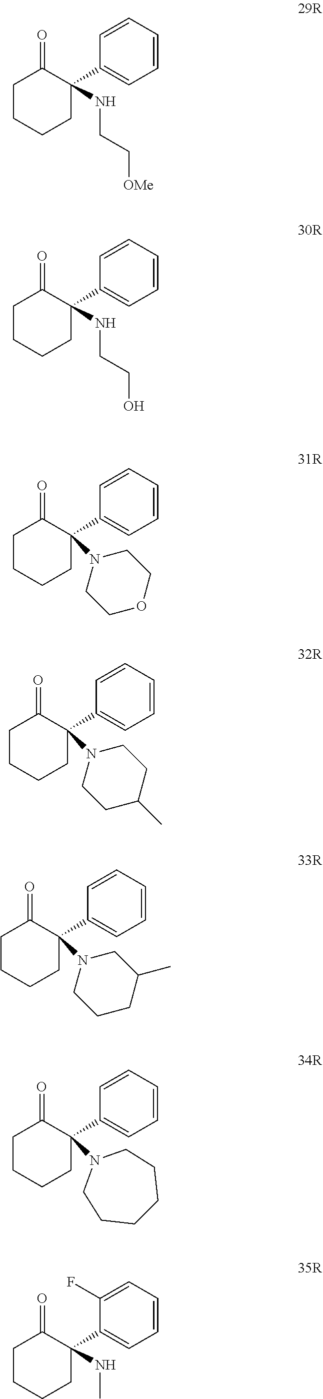

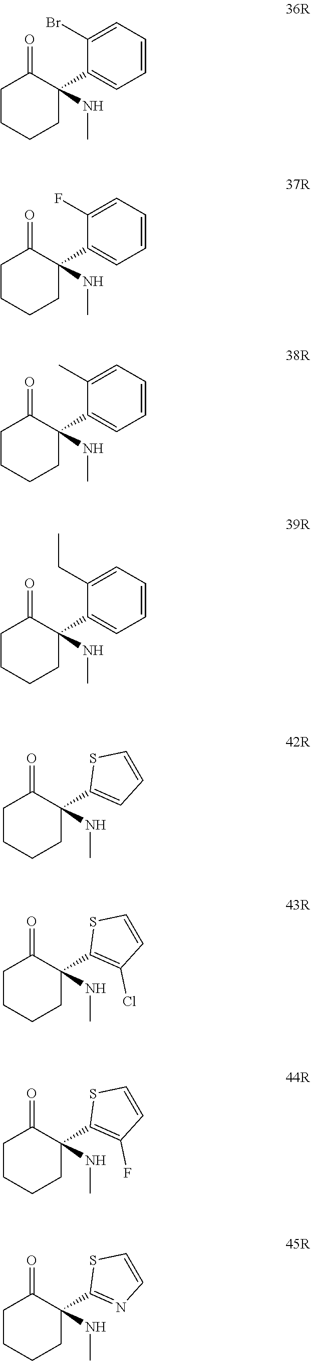

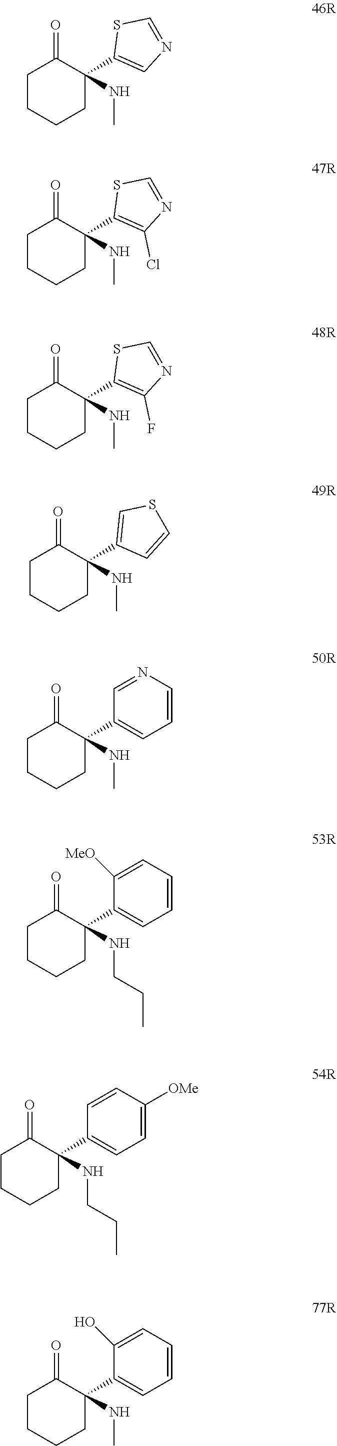

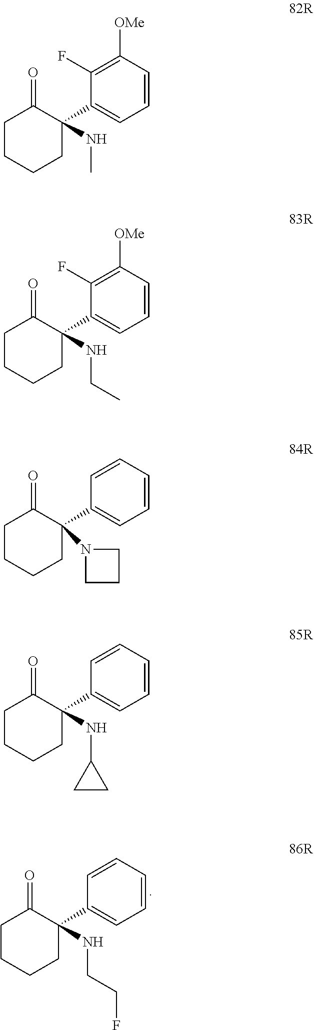 [SMILES: CN[C@@]1(c2cccc(OC)c2F)CCCCC1=O ; CCN[C@@]1(c2cccc(OC)c2F)CCCCC1=O ; O=C1CCCC[C@]1(c1ccccc1)N1CCC1 ; O=C1CCCC[C@@]1(NC1CC1)c1ccccc1 ; O=C1CCCC[C@@]1(NCCF)c1ccccc1]

In some embodiments, the NMDAR-ant compound is selected from:

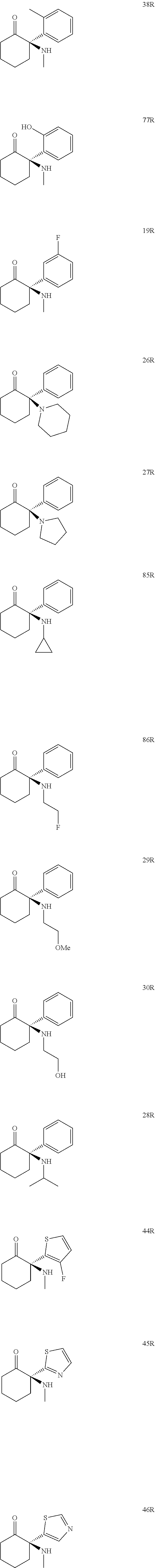

In some embodiments, the NMDAR-ant compound has the structure:

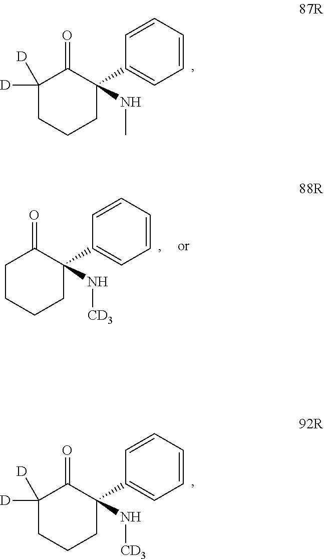

wherein D represents a deuterium-enriched H-site.

In some embodiments, each D represents a deuterium-enriched —H site and the level of deuterium at each deuterium-enriched —H site of the compound is 0.02% to 100%. In some embodiments, each D represents a deuterium-enriched —H site and the level of deuterium at each deuterium-enriched —H site of the compound is 20%-100%, 50%-100%, 70%-100%, 90%-100%, 97%-100%, or 99%-100%.

In some embodiments, the NMDAR-ant compound is selected from:

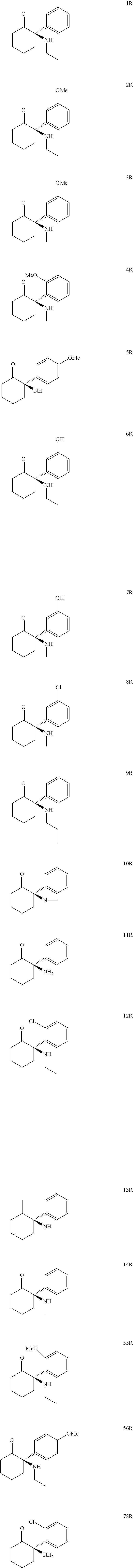

In some embodiments, the NMDAR-ant compound is selected from:

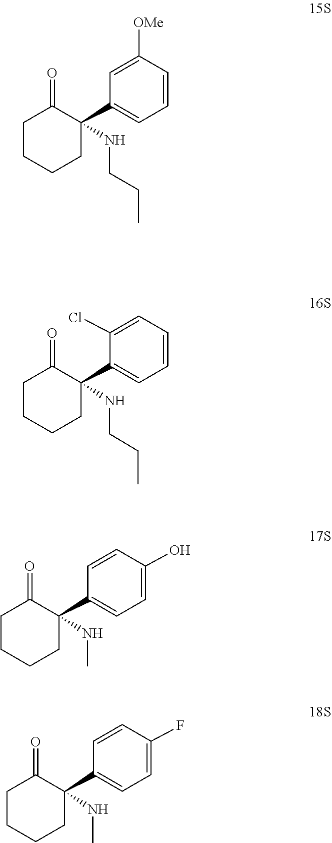 [SMILES: CCCN[C@]1(c2cccc(OC)c2)CCCCC1=O ; CCCN[C@]1(c2ccccc2Cl)CCCCC1=O ; CN[C@]1(c2ccc(O)cc2)CCCCC1=O ; CN[C@]1(c2ccc(F)cc2)CCCCC1=O]

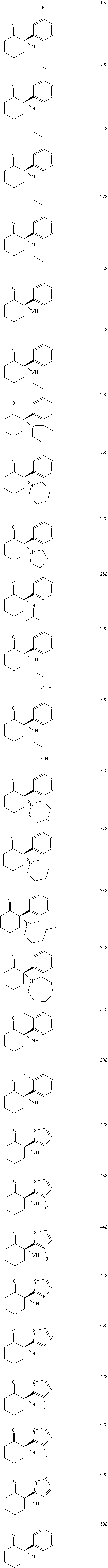

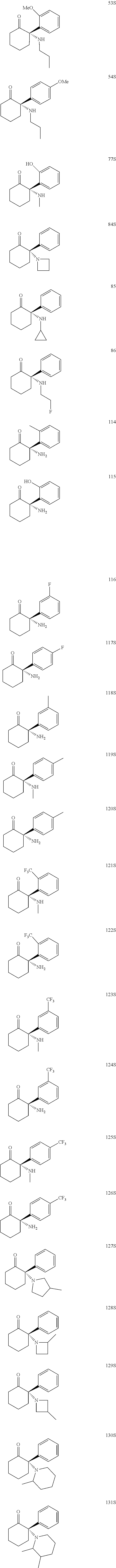

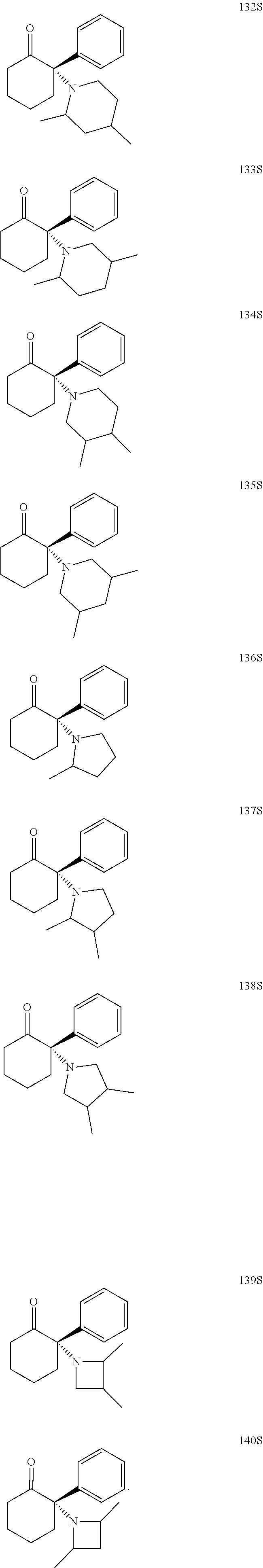

In some embodiments, the NMDAR-ant compound is selected from:

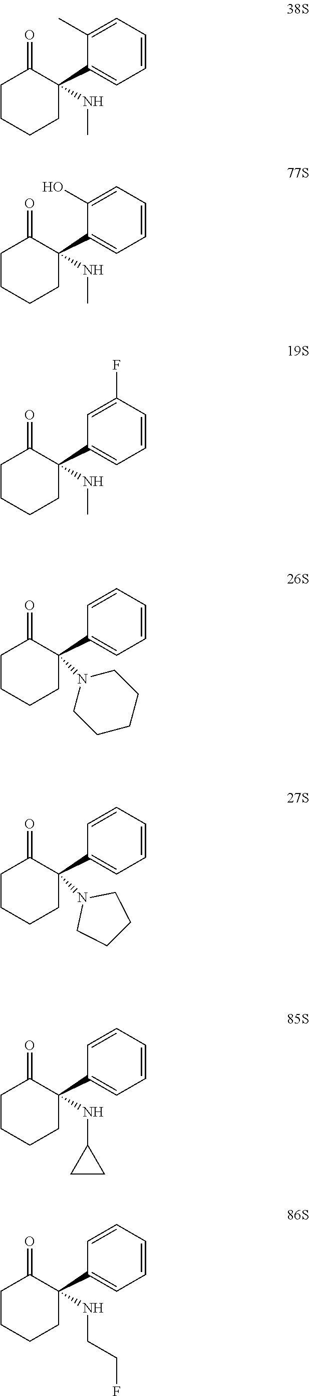

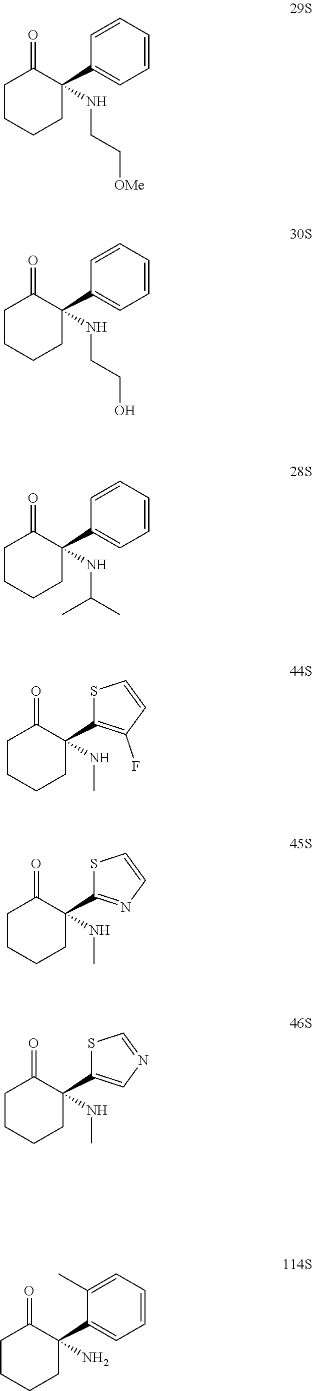

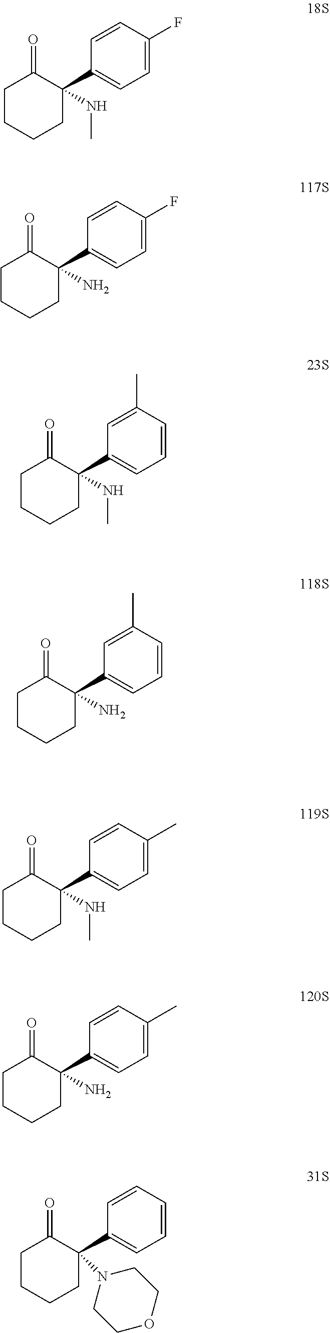

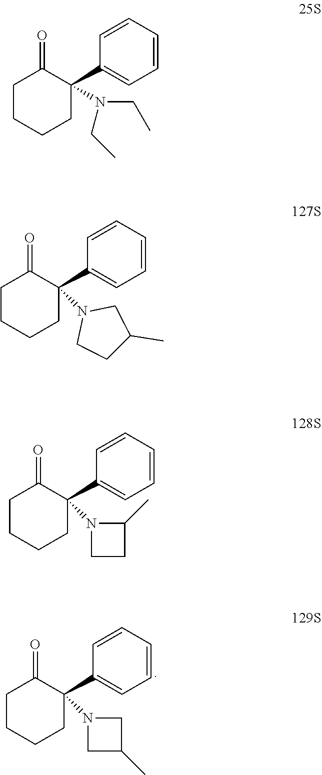 [SMILES: CCN(CC)[C@]1(c2ccccc2)CCCCC1=O ; CC1CCN([C@]2(c3ccccc3)CCCCC2=O)C1 ; CC1CCN1[C@]1(c2ccccc2)CCCCC1=O ; CC1CN([C@]2(c3ccccc3)CCCCC2=O)C1]

In some embodiments, the NMDAR-ant compound is selected from:

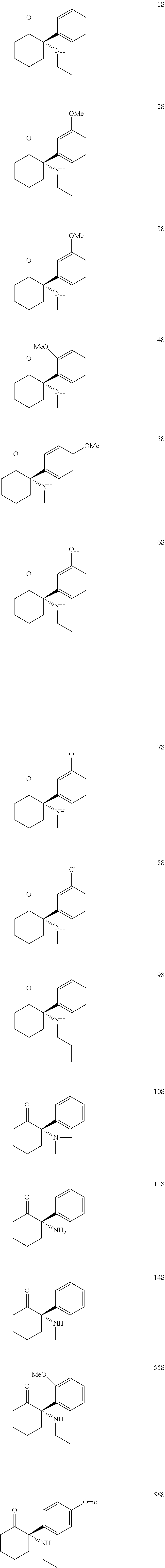

In some embodiments, the NMDAR-ant compound is selected from:

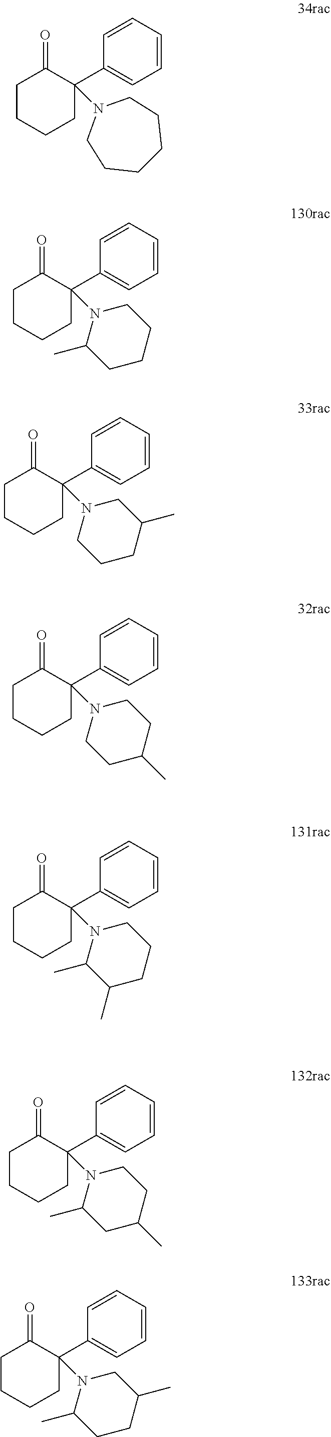

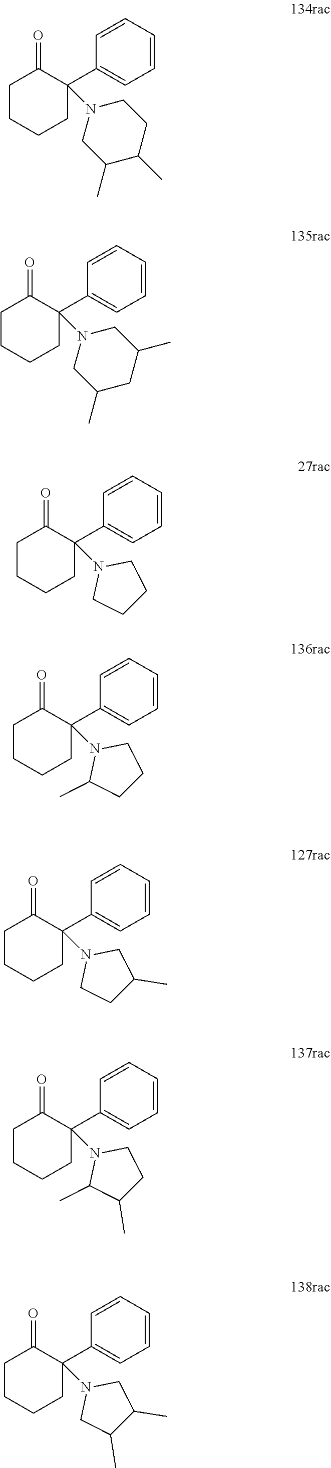

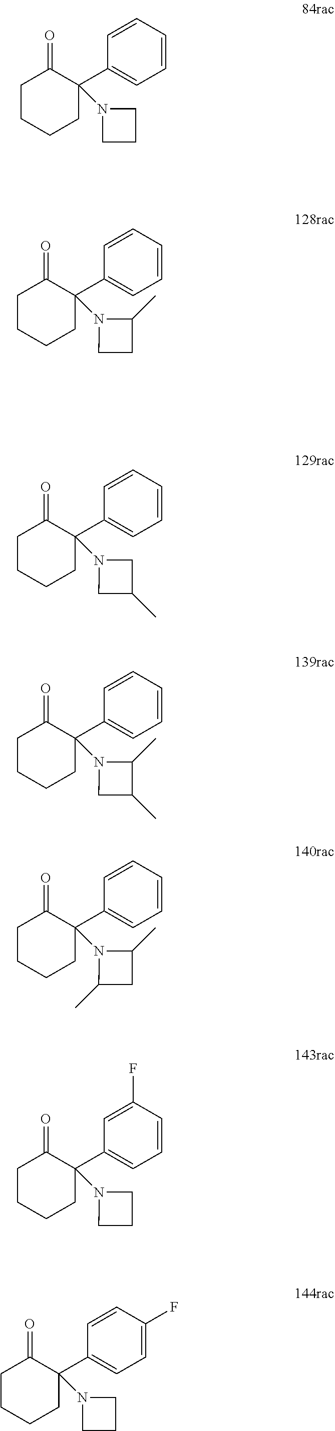

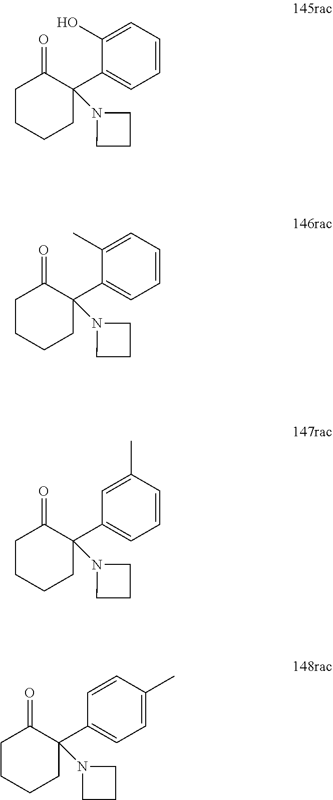 [SMILES: O=C1CCCCC1(c1ccccc1O)N1CCC1 ; Cc1ccccc1C1(N2CCC2)CCCCC1=O ; Cc1cccc(C2(N3CCC3)CCCCC2=O)c1 ; Cc1ccc(C2(N3CCC3)CCCCC2=O)cc1]

or a pharmaceutically acceptable salt or ester thereof, and/or a specific S- or R-isomer thereof.

In some embodiments, the NMDAR-ant compound is selected from:

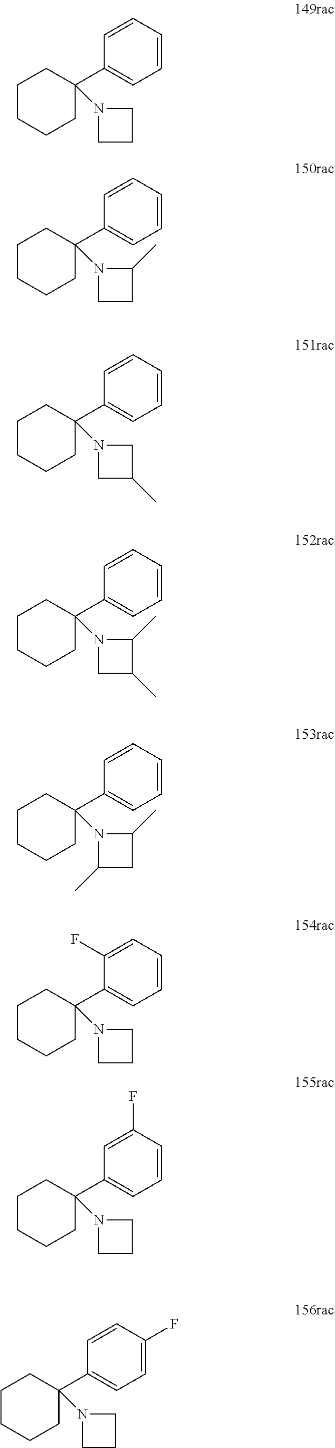

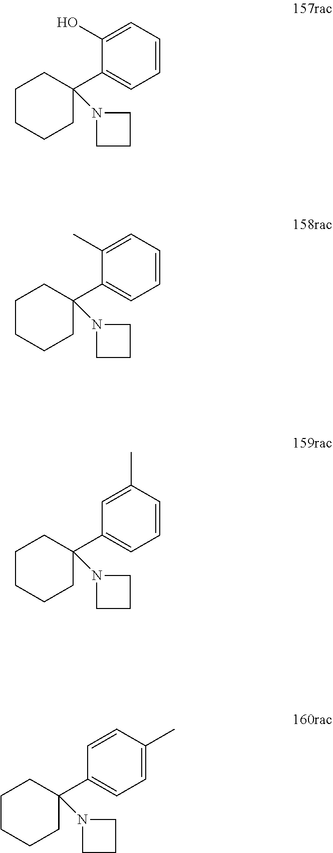 [SMILES: Oc1ccccc1C1(N2CCC2)CCCCC1 ; Cc1ccccc1C1(N2CCC2)CCCCC1 ; Cc1cccc(C2(N3CCC3)CCCCC2)c1 ; Cc1ccc(C2(N3CCC3)CCCCC2)cc1]

or a pharmaceutically acceptable salt or ester thereof, and/or a specific S- or R-isomer thereof.

In another aspect, the NMDAR-ant compound is an enantiomeric compound selected from the following:

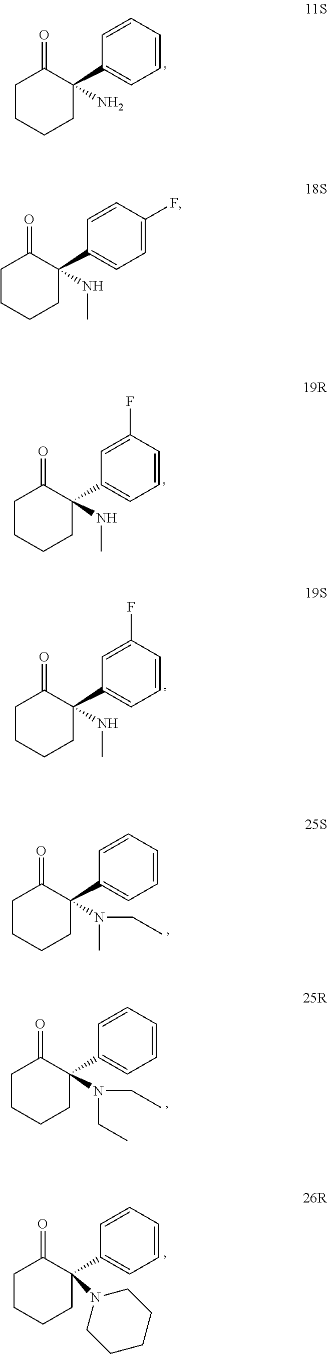

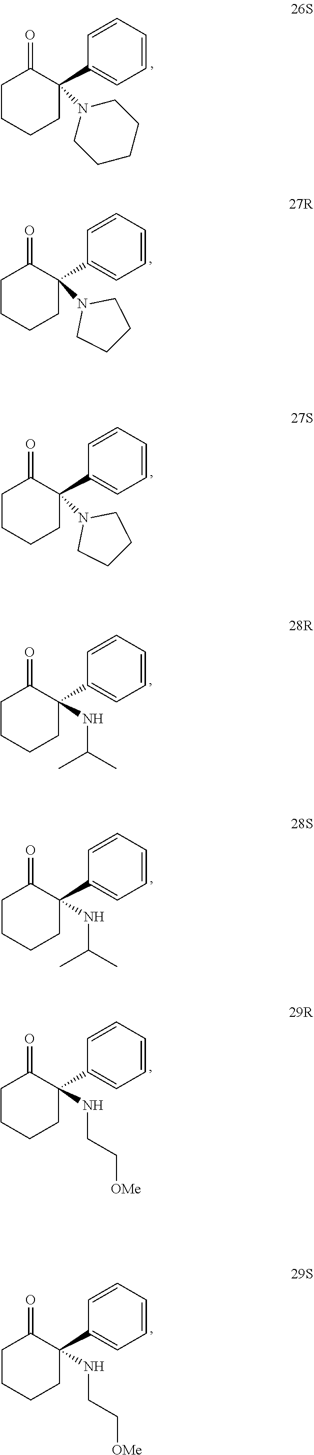

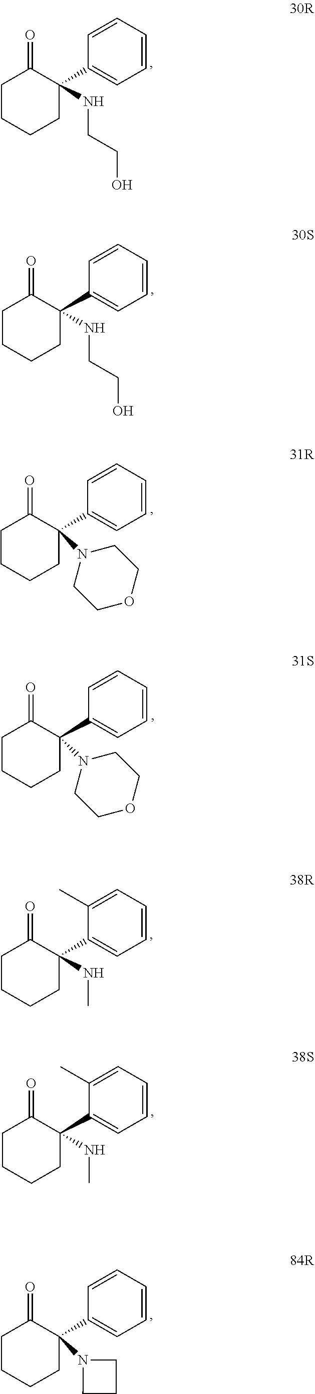

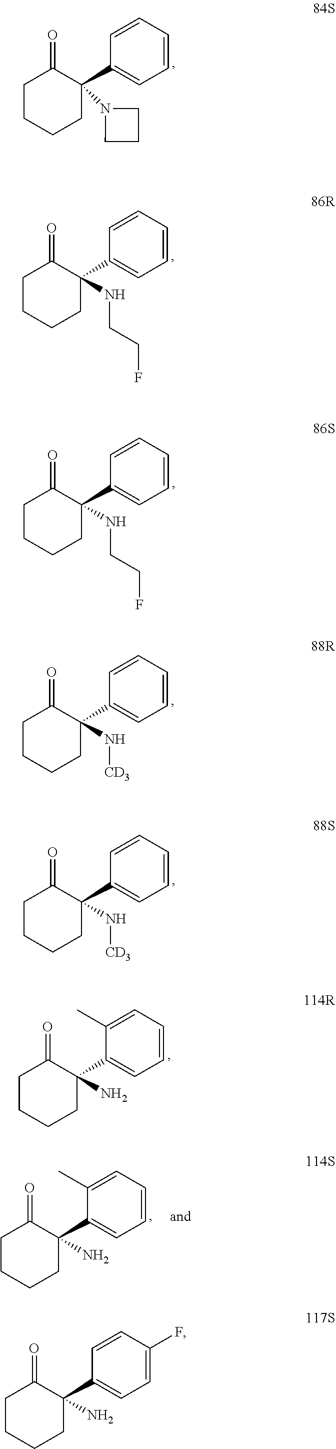

or a pharmaceutically acceptable salt thereof.

In some embodiments, the NMDAR-ant compound is an isolated, substantially enantiomerically pure compound selected from any of the above compounds, wherein the enantiomeric compound is present in an enantiomeric mixture having at least 90%, at least 95% or at least 99% of the enantiomeric compound.

In another aspect, the NMDAR-ant compound is selected from:

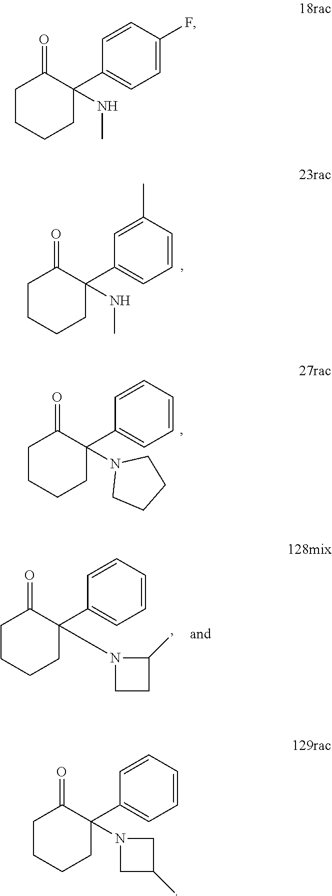

In some embodiments, the NMDAR-ant compound is selected from:

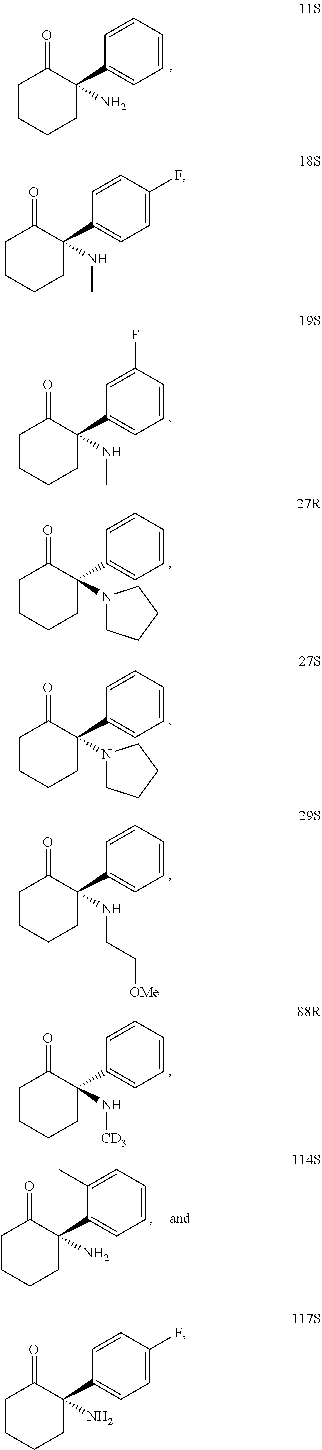

or a pharmaceutically acceptable salt thereof.

In some embodiments, the NMDAR-ant compound is an isolated, substantially enantiomerically pure compound selected from the group consisting of:

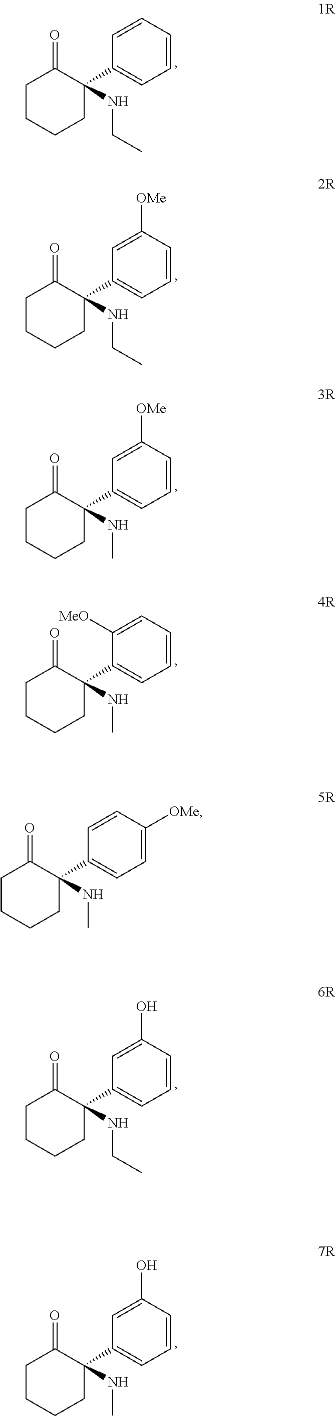

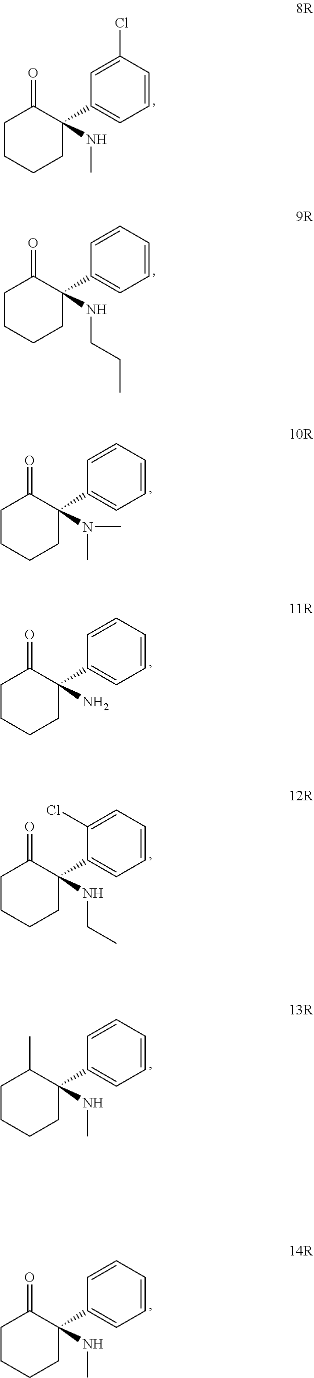

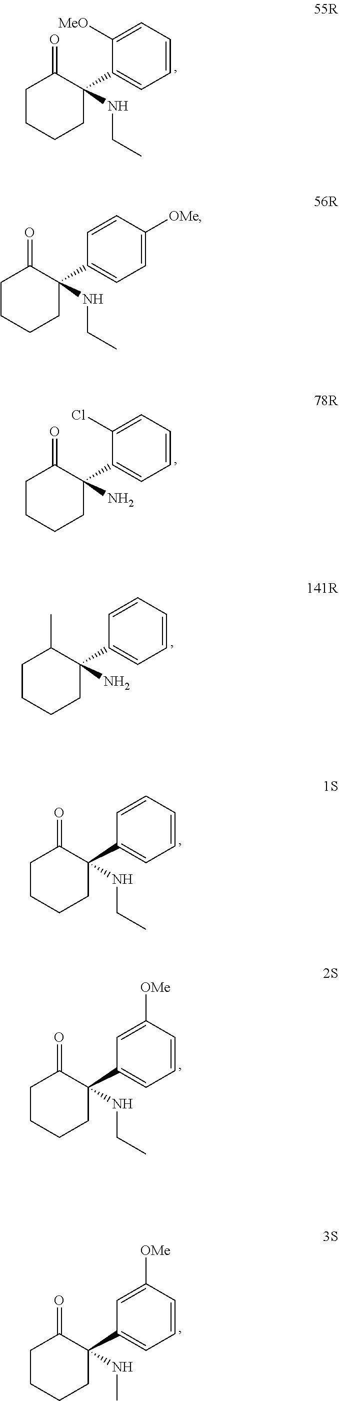

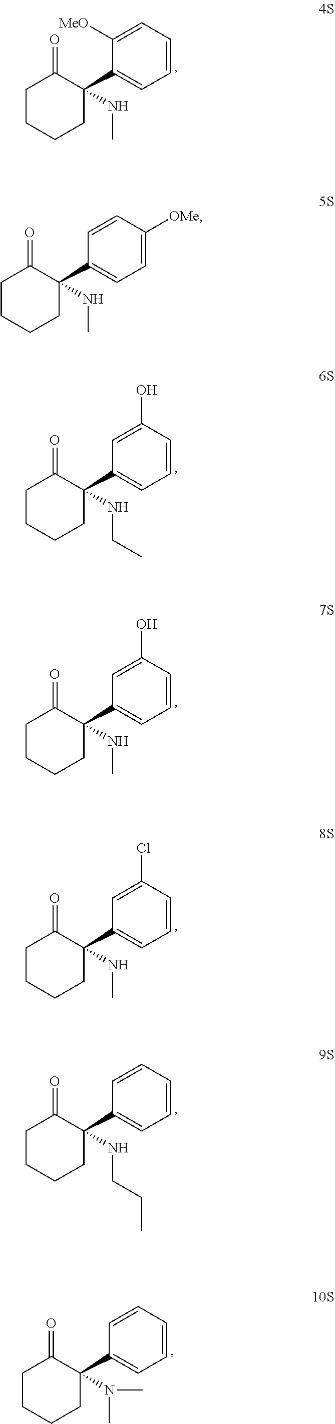

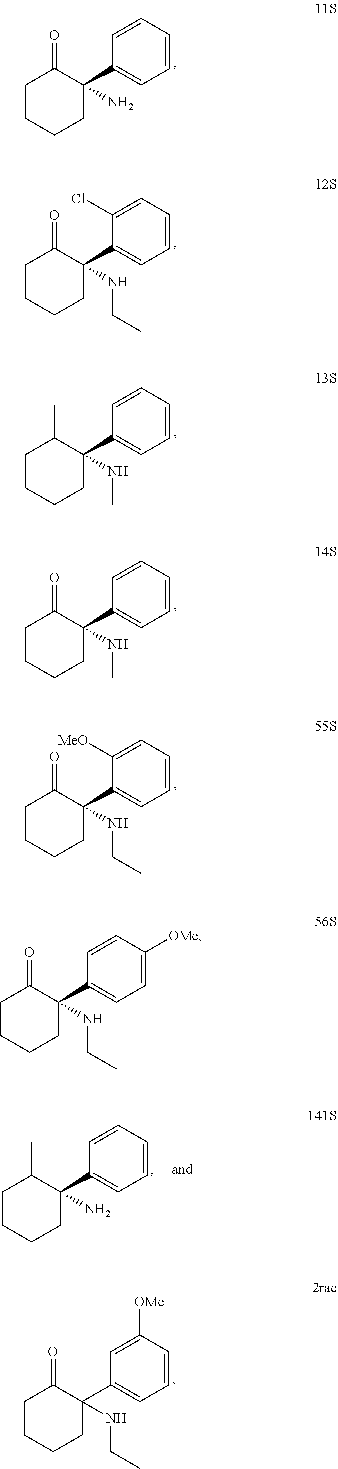

or a pharmaceutically acceptable salt thereof.

In some embodiments, the NMDAR-ant compound is selected from the following compounds:

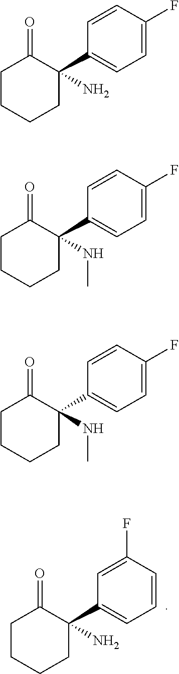 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]

Notably, any one or more of the foregoing NMDAR-ant compounds delineated above may be combined with any one or more of the AMPAR-PAM compounds delineated earlier above. All such combinations are considered herein. At the same time, one or more combinations may be excluded from the pharmaceutical composition.

In some embodiments, the optical purity of the AMPAR-PAM or NMDAR-ant compound is >5%, >25%, >50%, >75%, >90%, >95%, >97%, >98%, or >99%.

The present disclosure is also directed to pharmaceutical compositions containing the following components: (i) a positive allosteric modulator (PAM) of the AMPA receptor, or a pharmaceutically acceptable salt thereof; and (ii) (2R,3S)-2-amino-3-hydroxy-3-pyridin-4-yl-1-pyrrolidin-1-yl-propan-1-one, or a pharmaceutically acceptable salt thereof.

Component (i) can be any of the compounds described throughout this disclosure for component (i). In some embodiments, component (i) is PF-04958242 (BIIB-104; pesampator). In some embodiments, each dose unit of the composition contains between about 0.05 and about 1 mg of PF-04958242 (BIIB-104; pesampator). In some embodiments, each dose unit of the composition contains between about 0.1 and about 0.5 mg of PF-04958242 (BIIB-104; pesampator). In further or separate embodiments, each dose unit of the composition contains between about 10 and about 100 mg of component (ii). In further or separate embodiments, each dose unit of the composition contains between about 20 and about 40 mg of component (ii). In some embodiments, the composition further includes a pharmaceutically acceptable carrier, as amply described earlier above.

The term “C₁₋₁₀ alkyl”, as used in the present disclosure, refers to linear or branched saturated hydrocarbon groups (alkyl groups) containing one to ten carbon atoms. For any embodiment disclosed in the present disclosure, the C₁₋₁₀ alkyl may more particularly be selected as a C₂₋₁₀ alkyl, C₃₋₁₀ alkyl, C₂₋₁₀ alkyl, C₅₋₁₀ alkyl, C₆₋₁₀ alkyl, C₁₋₈ alkyl, C₂₋₈ alkyl, C₃₋₈ alkyl, C₄₋₈ alkyl, C₅₋₈ alkyl, C₆₋₈ alkyl, C₁₋₆ alkyl, C₂₋₆ alkyl, C₃₋₆ alkyl, C₄₋₆ alkyl, C₁₋₄ alkyl, C₂₋₄ alkyl, C₁₋₃ alkyl, or C₁₋₂ alkyl group. Some examples of linear alkyl groups include methyl, ethyl, n-propyl, n-butyl, n-pentyl, n-hexyl, n-heptyl, n-octyl, n-nonyl, and n-decyl groups. Some examples of branched alkyl groups include isopropyl (2-propyl), isobutyl (2-methylprop-1-yl), sec-butyl (2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, 1,2,2-trimethylprop-1-yl groups, and the numerous branched alkyl groups containing 7-10 carbon atoms, wherein the “1-yl” suffix represents the point of attachment of the group. The alkyl group may or may not contain (include) one or more heteroatoms as discussed above.

The term “C₂₋₁₀ alkenyl”, as used in the present disclosure, refers to linear, branched, or cyclic (C₃₋₇ cycloalkenyl) hydrocarbon groups containing one to ten carbon atoms and at least one carbon-carbon double bond. For any embodiment disclosed in the present disclosure, the C₂₋₁₀ alkenyl group may more particularly be selected as a C₃₋₁₀ alkenyl, C₄₋₁₀ alkenyl, C₅₋₁₀ alkenyl, C₆₋₁₀ alkenyl, C₂₋₈ alkenyl, C₃₋₈ alkenyl, C₄₋₈ alkenyl, C₅₋₈ alkenyl, C₆₋₈ alkenyl, C₂₋₆ alkenyl, C₃₋₆ alkenyl, C₄₋₆ alkenyl, C₂₋₄ alkenyl, or C₂₋₃ alkenyl group. Some examples of linear C₂₋₁₀ alkenyl groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, and 6-hepten-1-yl. Some examples of branched C₂₋₁₀ alkenyl groups include 2-propen-2-yl, 3-buten-2-yl, 3-buten-3-yl, 4-penten-2-yl, 4-penten-3-yl, 3-penten-2-yl, 3-penten-3-yl, 2,4-pentadien-3-yl, and the numerous C₆, C₇, C₈, C₉, and C₁₀ branched alkenyl groups. In the case of a C₃₋₇ cycloalkenyl group, the group includes aliphatic but not aromatic groups (e.g., cyclopropenyl, cyclobutenyl, cyclopentenyl, cyclopentadienyl, cyclohexenyl, and cyclohexadienyl). The alkenyl group may or may not contain (include) one or more heteroatoms as discussed above.

The term “C₂₋₁₀ alkynyl”, as used in the present disclosure, refers to linear or branched hydrocarbon groups containing one to ten carbon atoms and at least one carbon-carbon triple bond. For any embodiment disclosed in the present disclosure, the C₂₋₁₀ alkynyl group may more particularly be selected as a C₃₋₁₀ alkynyl, C₄₋₁₀ alkynyl, C₅₋₁₀ alkynyl, C₆₋₁₀ alkynyl, C₂₋₈ alkynyl, C₃₋₈ alkynyl, C₄₋₈ alkynyl, C₅₋₈ alkynyl, C₆₋₈ alkynyl, C₂₋₆ alkynyl, C₃₋₆ alkynyl, C₄₋₆ alkynyl, C₂₋₄ alkynyl, or C₂₋₃ alkynyl group. Some examples of linear C₂₋₁₀ alkynyl groups include ethynyl, propargyl, 2-butynyl, 3-butynyl, and 4-pentynyl groups. The alkynyl group may or may not contain (include) one or more heteroatoms as discussed above.

The term “C₃₋₇ cycloalkyl”, as used in the present disclosure, refers to cyclic hydrocarbon groups containing three to seven carbon atoms. The term “C₃₋₁₀ cycloalkyl”, as used in the present disclosure, refers to cyclic hydrocarbon groups containing three to ten carbon atoms. Some examples of C₃₋₇ cycloalkyl groups include cyclopropyl, 1-methylenecyclopropyl (i.e., with presence of a methylene linker), 2-methylcycloprop-1-yl, 2,3-dimethylcycloprop-1-yl, cyclobutyl, 1-methylenecyclobutyl, 2-methylcyclobut-1-yl, 2,4-dimethylcyclobut-lyl, cyclopentyl, 1-methylenecyclopentyl, 2-methylcyclopent-1-yl, 2,5-dimethylcyclopent-1-yl, 3,4-dimethylcyclopent-1-yl, cyclohexyl, 1-methylenecyclohexyl, 2-methylcyclohex-1-yl, 2,6-dimethylcyclohex-1-yl, 3,5-dimethylcyclohex-1-yl, and cycloheptyl groups. In some embodiments, the C₃₋₇ cycloalkyl group is carbocyclic. In other embodiments, the C₃₋₇ cycloalkyl group may also include (i.e., contain) one or more heteroatoms (e.g., N, O, and/or S) in the ring, in which case the hydrocarbon group is a C₃₋₇ heterocycloalkyl group. Some examples of C₃₋₇ heterocycloalkyl groups include piperidinyl, piperazinyl, tetrahydrofuranyl, and dioxolanyl groups. The term “cycloalkyl”, as used herein, also includes bicyclic ring systems in which one or both rings are cycloalkyl, e.g., decalinyl. The term “cycloalkyl” is also intended (unless otherwise specified) to include groups containing a cycloalkyl ring connected to a non-cyclic group, such as an alkyl group, in which case the group may be further specified as an alkyl-cycloalkyl group or cycloalkyl-alkyl group, such as methyl-cyclohexyl (tolyl). The cycloalkyl group may or may not contain (include) one or more heteroatoms as discussed above.

The term “aryl,” as used in the present disclosure, refers to the numerous carbocyclic hydrocarbon groups that are aromatic. The aryl group may be monocyclic or bicyclic. Some examples of monocyclic aryl groups include phenyl and tolyl groups. Some examples of bicyclic aryl groups include naphthalene, indanyl, and indenyl groups.

The term “heteroaryl,” as used in the present disclosure, refers to the numerous heterocyclic hydrocarbon groups that are aromatic. The heteroaryl group may be monocyclic or bicyclic. Some examples of monocyclic heteroaryl groups include pyridinyl, pyrazinyl, pyrrolyl, pyrimidinyl, and thienyl groups. Some examples of bicyclic heteroaryl groups include indolyl and purinyl groups.

Compounds disclosed herein may include at least one asymmetric center. These centers are designated by the symbols “R” or “S,” depending on the configuration of substituents around the chiral atom. Unless otherwise indicated in the structural formula, it should be understood that the invention encompasses all stereochemical isomeric forms, including diastereomeric, enantiomeric, and epimeric forms, as well as d-isomers and 1-isomers, and mixtures thereof, for any compounds disclosed in the present disclosure. Individual stereoisomers of compounds can be prepared synthetically from commercially available starting materials which contain chiral centers or by preparation of mixtures of enantiomeric products followed by separation, such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, any of the presently disclosed compounds may exist as tautomers; all tautomeric isomers are considered in this disclosure. Additionally, any of the compounds disclosed herein can exist in unsolvated as well as solvated forms with pharmaceutically acceptable solvents such as water, ethanol, and the like.

In some embodiments, a compound disclosed herein may be enriched in a specific enantiomer relative to the corresponding opposite enantiomer of that compound, such that the mixture is not racemic. In such cases, the subject mixture of isomers is understood to have an enantiomeric excess and optical purity >0%. The enantiomeric excess or optical purity of the isomeric mixture may be, for example, >0%, >5%, >25%, >50%, >75%, >90%, >95%, >97%, >98%, or >99%. The enantiomeric excess or optical purity of the isomeric mixture may be, for example, 5-100%, 25-100%, 50-100%, 75-100%, 90-100%, 95-100%, 97-100%, 98-100%, or 99-100%. Thus, for example, a compound disclosed herein may include the S enantiomer of a compound substantially free of the R enantiomer, or the R enantiomer substantially free of the S enantiomer. Further, if the named compound includes more than one chiral center, the scope of the present disclosure also includes compositions including mixtures of varying proportions between the diastereomers, as well as compositions including one or more diastereomers substantially free of one or more of the other diastereomers. By “substantially free” it is meant that the composition includes less than 50%, 25%, 15%, 10%, 8%, 5%, 3%, 2%, or 1% of the minor enantiomer or diastereomer(s).

For clarity, in the context of the present disclosure, the chemical structure of a compound depicted with a specific stereochemical orientation at any particular chiral center, as defined by wedge and dash notation, is intended to represent the indicated stereoisomer of the compound in substantially pure form, or a mixture enriched in the stereoisomer(s) with the indicated stereochemical orientation at the defined chiral center over the stereoisomer(s) with the opposite orientation at the chiral center.

Any of the compounds disclosed herein may be in the form of a pharmaceutically acceptable salt thereof. The phrase “pharmaceutically acceptable salt(s)”, as used herein, refers to those salts of compounds disclosed herein that are safe and effective for pharmaceutical use in mammals and that retain the desired biological activity.

Pharmaceutically acceptable salts include salts of acidic or basic groups present in compounds disclosed herein. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds disclosed herein can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see Berge et al., 66 J. Pharm. Sci. 1-19 (1977), incorporated herein by reference.

The compounds disclosed herein may also include one or more isotopes of atoms occurring on the compounds disclosed herein. Isotopes include those atoms having the same atomic number but different mass numbers. By way of general example and without limitation, isotopes of hydrogen include tritium and deuterium, and isotopes of carbon include ¹³C and ¹⁴C.

In another aspect, the invention is directed to pharmaceutical compositions that contain the above-described components (i) and (ii) dispersed in a pharmaceutically acceptable carrier, i.e., vehicle or excipient. The components are dispersed in the pharmaceutically acceptable carrier by either being mixed, either in solid form with a solid carrier or dissolved or emulsified in a liquid carrier. The pharmaceutical composition may or may not also be formulated together with one or more additional active ingredients or adjuvants that improve the overall efficacy of the pharmaceutical composition.

Components (i) and (ii) and the carrier may be formulated into pharmaceutical compositions and dosage forms according to methods well known in the art. The pharmaceutical compositions of the present invention may be formulated for administration in liquid or solid form. In different embodiments, the pharmaceutical formulation may be formulated for oral administration (e.g., as tablets, capsules, powders, granules, pastes, solutions, suspensions, drenches, or syrups); parenteral administration (e.g., by subcutaneous, intramuscular or intravenous injection as provided by, for example, a sterile solution or suspension); topical application (e.g., as a cream, ointment, or spray); sublingual or buccal administration; ocular administration; transdermal administration; or nasal administration.

Pharmaceutical compositions suitable for oral administration may be presented as discrete dosage units such as pills, tablets, dragées, or capsules, or as a powder or granules, or as a solution or suspension. The active ingredient may also be presented as a bolus or paste. Tablets may contain the active ingredient compounds and suitable binders, lubricants, disintegrating agents, coloring agents, flavoring agents, flow-inducing agents, and melting agents. Gelatin capsules may contain the active ingredient compounds and powdered carriers, such as lactose, starch, cellulose derivatives, magnesium stearate, stearic acid, and the like. Similar diluents can be used to make compressed tablets. Compressed tablets can be sugar-coated or film-coated to mask an unpleasant taste and protect the tablet from the atmosphere, or enteric-coated for selective disintegration in the gastrointestinal tract. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, for example, starch, methyl cellulose, agar, bentonite, xanthan gum, and the like.

For oral administration in liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Liquid dosage forms for oral administration may contain coloring and flavoring to increase patient acceptance.

For parenteral administration, suitable compositions may include aqueous and non-aqueous sterile solutions. In general, water, a suitable oil, saline, aqueous dextrose (glucose), and related sugar solutions and glycols, such as propylene glycol or polyethylene glycols, are suitable carriers for parenteral solutions. Solutions for parenteral administration preferably contain a water-soluble salt of the active ingredient, suitable stabilizing agents, and if necessary, buffer substances. Anti-oxidizing agents, such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. The compositions may be presented in unit-dose or multi-dose containers, for example, sealed vials and ampoules, and may be stored in a freeze-dried (lyophilized) condition requiring only the addition of sterile liquid carrier, for example water, prior to use. For transdermal administration, e.g. gels, patches or sprays can be contemplated. Compositions or formulations suitable for pulmonary administration, e.g., by nasal inhalation, include fine dusts or mists which may be generated by means of metered dose pressurized aerosols, nebulizers or insufflators. Parenteral and intravenous forms may also include minerals and other materials to make them compatible with the type of injection or delivery system chosen.

Any of the compounds or combinations thereof disclosed in the present disclosure may also be administered in the form of a liposome delivery system, such as a small unilamellar vesicle, large unilamellar vesicle, or multilamellar vesicle. Liposomes, as well as known in the art, can be formed from a variety of phospholipids, such as cholesterol, stearylamine, or phosphatidylcholines. The compounds may be administered as components of tissue-targeted emulsions.

Any of the compounds or combinations thereof disclosed in the present disclosure may also be coupled to targetable drug carriers, such as soluble polymers. Such polymers include, for example, polyvinylpyrrolidone, pyran copolymer, polyhydroxylpropylmethacrylamide, polyhydroxyethylaspartamide, and polyethyleneoxide-polylysine substituted with palmitoyl residues. The compounds may also be coupled to a class of biodegradable polymers useful in achieving controlled release of a drug, such as, for example, polylactic acid, polyglycolic acid, copolymers of polylactic and polyglycolic acid, polycaprolactone, polyhydroxy butyric acid, polyorthoesters, polyacetals, polydihydropyrans, polycyanoacrylates, and crosslinked or amphipathic block copolymers of hydrogels.

Pharmaceutical compositions disclosed herein may, in some embodiments, be provided with immediate release, delayed release, extended release, or modified release profiles. In some embodiments, pharmaceutical compositions with different drug release profiles may be combined to create a two-phase or three-phase release profile. For example, pharmaceutical compositions may be provided with an immediate release and an extended release profile. In some embodiments, pharmaceutical compositions may be provided with an extended release and delayed release profile. Such compositions may be provided as pulsatile formulations, multilayer tablets, or capsules containing tablets, beads, granules, etc. Pharmaceutical compositions herein may also be provided with abuse deterrent features by techniques know in the art, such as, for example, by making a tablet that is difficult to crush or to dissolve in water. The invention further includes a pharmaceutical composition, as herein described, in combination with a packaging material, including instructions for the use of the composition for a use as herein described. In some embodiments, any of the compounds disclosed in the present disclosure may alternatively be in the form of a prodrug.

The phrase “pharmaceutically acceptable” refers herein to those compounds, materials, compositions, and/or dosage forms which are, within the scope of sound medical judgment, suitable for administration to a subject. The phrase “pharmaceutically acceptable carrier,” as used herein, refers to a pharmaceutically-acceptable vehicle, such as a liquid or solid filler, diluent, carrier, manufacturing aid (e.g., lubricant, talc magnesium, calcium or zinc stearate, or stearic acid), solvent, or encapsulating material, that serves to carry the therapeutic composition for administration to the subject. Each carrier should be “acceptable” in the sense of being compatible with the other ingredients of the formulation and physiologically safe to the subject. Any of the carriers known in the art can be suitable herein depending on the mode of administration.

Some examples of materials that can serve as pharmaceutically-acceptable excipients, particularly for liquid forms, include water; isotonic saline; pH buffering agents; sugars (e.g., lactose, glucose, sucrose, and oligosaccharides, such as sucrose, trehalose, lactose, or dextran); and antimicrobials. Other excipients, more typically used in solid dosage forms, may also be included, e.g., starches (e.g., corn and potato starch); cellulose and its derivatives (e.g., sodium carboxymethyl cellulose, ethyl cellulose and cellulose acetate); gelatin; talc; waxes; oils (e.g., peanut oil, cottonseed oil, safflower oil, sesame oil, olive oil, corn oil and soybean oil); glycols (e.g., ethylene glycol, propylene glycol, and polyethylene glycol); polyols (e.g., glycerin, sorbitol, and mannitol); esters (e.g., ethyl oleate and ethyl laurate); agar; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

In some embodiments, the carrier further includes a molecular or microscopic (e.g., microscale or nanoscale) sub-carrier in which the pharmaceutical composition or component thereof is loaded, either within and/or conjugated onto the surface of the sub-carrier. The sub-carrier can be composed of, for example, a biocompatible and biodegradable polymer, e.g., based on a polyhydroxyacid biopolyester or polysaccharide. The overall structure of the sub-carrier can be, for example, a micelle, a liposome, dendrimer, nanoparticle, or porous scaffold. These and numerous other types of sub-carriers are well known in the art.

The sub-carrier may function to protect the pharmaceutical composition or component thereof during transit, e.g., while in the bloodstream or while passing through the gastrointestinal tract. The sub-carrier may also function to release the pharmaceutical composition or component thereof closer to target cells with a lower chance of degradation, if applicable. The sub-carrier may also be functionalized with one or more targeting agents that selectively target a class of cells to be treated with the pharmaceutical composition or component thereof, if applicable. The targeting agent can be, for example, an antibody, antibody fragment, siRNA, or small molecule receptor binder.

In another aspect, the invention is directed to a method for treating a mental disorder by administering to a subject having the mental disorder a pharmaceutically effective amount of components (i) and (ii). As further discussed below, components (i) and (ii) may be administered together at the same time or separately at different times provided that the duration between administrations of components (i) and (ii) permits component (i) to modulate the effect of component (ii) in the subject and/or vice-versa. The term “mental disorder” includes any adverse or undesirable mental condition, and is thus intended to include any of the known mental conditions, psychiatric disorders, and psychiatric conditions, such as any of those catalogued in the Diagnostic and Statistical Manual of Mental Disorders (DSM-5). The mode of administration may be any of the modes of administration described above. A typical mode of administration for purposes of the present invention may be oral or intravenous injection.

In the method of treatment, components (i) and (ii) are administered in a therapeutically effective amount to a subject (patient). The patient is typically human, but may in some cases be a non-human mammal, such as a dog, cat, primate, or cattle. The therapeutically effective amount of each component is an amount that results in mitigation of one or more symptoms of the mental disorder or condition being treated. The therapeutically effective amount for each component can be readily determined according to methods familiar to physicians and clinicians, e.g., during pre-clinical and clinical trials. As is well known in the art, the dosage of the active ingredient(s) depends not only on the type of mental disorder or condition being treated, but the method of administration, size of the patient, and potential side effects. Dosing is dependent on the severity and responsiveness of the mental disorder or condition being treated, with the course of treatment lasting from several days to several months, or until an acceptable mitigation of one or more symptoms of the mental disorder or condition is achieved. Optimal dosing schedules can be calculated from measurements of drug accumulation in the body of the patient. The administering physician can determine optimum dosages, dosing methodologies, and repetition rates.

More particularly, the terms “effective amount” or “therapeutically effective amount” refer to an amount of a compound, such as any of compounds (i) and (ii) or combination thereof as described above, effective for diminishing or preventing a system of a mental condition or disorder. The effective amount may function to diminish, for example: depression, anxiety, OCD, substance addiction, personality disorder, sadness or lethargy, depressed mood, anxious or sad feelings, diminished interest in all or nearly all activities, significant increased or decreased appetite leading to weight gain or weight loss, insomnia, irritability, fatigue, feelings of worthlessness, feelings of helplessness, inability to concentrate, or recurrent thoughts of death or suicide. The effective amount may also function to provide a desired pharmacologic and/or physiologic effect, for example, reducing, inhibiting, or reversing one or more of the underlying pathophysiological mechanisms underlying the neurological dysfunction, modulating dopamine levels or signaling, modulating serotonin levels or signaling, modulating norepinephrine levels or signaling, modulating glutamate or GABA levels or signaling, modulating synaptic connectivity or neurogenesis in certain brain regions, or a combination thereof.

The term “therapeutic index” used in reference to any compound and its associated therapeutic effects and side effects refers to the ratio of the dose of said compound required to induce a particular negative side effect to the dose of said compound required to induce the desired therapeutic effect. For example, in the case of racemic ketamine, antidepressant therapeutic effects and dissociative side effects occur at similar doses and thus, the therapeutic index of this compound in this context is about 1:1. In contrast, a compound or combination of components (i) and (ii) disclosed herein may have an improved therapeutic index, for example, about 3:1, where a 3-fold higher dose is required to induce dissociative side effects relative to that needed for antidepressant therapeutic effects.

The terms “about” or “approximately,” as used herein, indicate an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range up to 10%, a range up to 5%, and/or a range up to 1% of a given value. Alternatively, particularly with respect to biological systems or processes, the term can mean within an order of magnitude, e.g., within 5-fold, or within 2-fold, of a value. “About” and “approximately” are used interchangeably herein.

In different embodiments, depending on various factors discussed above, a suitable dosage of the components (i) and/or (ii) or combination thereof may independently be precisely, at least, no more than, or less than, for example, 1 mg, 2 mg, 3 mg, 4 mg, 5 mg, 6 mg, 7 mg, 8 mg, 9 mg, 10 mg, 15 mg, 20 mg, 25 mg, 30 mg, 40 mg, 50 mg, 100 mg, 200 mg, 300 mg, 400 mg, 500 mg, 600 mg, 700 mg, 800 mg, 900 mg, 1000 mg, 1200 mg, or 1500 mg, per kg or per 50 kg, 60 kg, or 70 kg adult, or a dosage within a range bounded by any of the foregoing exemplary dosages. Depending on these and other factors, any of components (i) and (ii), as described above, are independently administered in the indicated dosage by any suitable schedule, e.g., once, twice, or three times a day, or once every two days, or once every three days, or once weekly, or once biweekly, twice weekly, once monthly, twice monthly, or three times monthly, for a total treatment time of one, two, three, four, or five days, and up to, for example, one, two, three, or four weeks, or one, two, three, four, five, six months, a year, or longer. The indicated dosage may alternatively be administered every two or three days, or per week. Alternatively, or in addition, the composition is administered until a desired change is evidenced.

In some embodiments, methods include treating a mental or psychiatric disorder or condition by administering to a subject in need thereof about 0.01 mg to about 400 mg of compound (i) and/or compound (ii) or combination thereof, wherein compounds (i) and (ii) may be administered separately or in the form of a pharmaceutical composition in which compounds (i) and (ii) are both included, as described above. In some embodiments, a dosage of compound (i) and/or compound (ii) or combination thereof or a pharmaceutically acceptable salt thereof may be in a range of, e.g., about 0.1 to 300 mg, 0.1 to 250 mg, 0.1 to 200 mg, 0.1 to 150 mg, 0.1 to 100 mg, 0.1 to 75 mg, 0.1 to 50 mg, 0.1 to 25 mg, 0.1 to 20 mg, 0.1 to 15 mg, 0.1 to 10 mg, 0.1 to 5 mg, 0.1 to 1 mg, 10 to 300 mg, 10 to 250 mg, 10 to 200 mg, 10 to 150 mg, 10 to 100 mg, 10 to 50 mg, 10 to 25 mg, 10 to 15 mg, 20 to 300 mg, 20 to 250 mg, 20 to 200 mg, 20 to 150 mg, 20 to 100 mg, 20 to 50 mg, 50 to 300 mg, 50 to 250 mg, 50 to 200 mg, 50 to 150 mg, 50 to 100 mg, 100 to 300 mg, 100 to 250 mg, 100 to 200 mg, with doses of, e.g., about 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 10 mg, 15 mg, 20 mg, 25 mg, 30, mg, 35 mg, 40 mg, 45 mg, 50 mg, 75 mg, 100 mg, 125 mg, 150 mg, 175 mg, 200 mg, 225 mg, 250 mg, 275 mg, 300 mg, and 400 mg being examples.

In some embodiments, dosages may include amounts of compound (i) and/or compound (ii) or combination thereof or a pharmaceutically acceptable salt thereof in the range of about, e.g., 1 mg to 200 mg, 1 mg to 100 mg, 1 mg to 50 mg, 1 mg to 40 mg, 1 mg to 30 mg, 1 mg to 20 mg, 1 mg to 15 mg, 0.01 mg to 10 mg, 0.1 mg to 15 mg, 0.15 mg to 12.5 mg, or 0.2 mg to 10 mg, with doses of 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12 mg, 15 mg, 20 mg, 25 mg, 30 mg, 35 mg, 40 mg, 45 mg, 50 mg, 60 mg, 75 mg, 80 mg, 90 mg, 100 mg, 125 mg, 150 mg, and 200 mg being specific examples of doses.

In some embodiments, component (i) is or includes a biarylpropylsulfonamide, and the biarylpropylsulfonamide may be or include PF-04958242 (BIIB-104; pesampator). In some embodiments, each dose unit of the composition contains between about 0.05 and about 1 mg of PF-04958242 (BIIB-104; pesampator). In some embodiments, each dose unit of the composition contains between about 0.1 and about 0.5 mg of PF-04958242 (BIIB-104; pesampator).

Dosages of compound (i) and/or compound (ii) or combination thereof or a pharmaceutically acceptable salt thereof, may be administered, for example, once, twice, three or four times daily, every other day, every three days, once weekly, or once a month to a patient in need thereof. In some embodiments, the dosage is about, e.g., 1-400 mg/day, or 1-300 mg/day, or 1-250 mg/day, or 1-200 mg/day, for example 300 mg/day, 250 mg/day, 200 mg/day, 150 mg/day, 100 mg/day, 75 mg/day, 50 mg/day, 25 mg/day, 20 mg/day, 10 mg/day, 5 mg/day, or 1 mg/day.

In some embodiments, pharmaceutical compositions for parenteral or inhalation, e.g., a spray or mist of the pharmaceutical composition of the present invention or a pharmaceutically acceptable salt thereof, include a concentration of about 0.005 mg/mL to about 500 mg/mL for compound (i), compound (ii), or combination thereof. In some embodiments, the compositions include compound (i) and/or compound (ii) or combination thereof or a pharmaceutically acceptable salt thereof at a concentration of, e.g., about 0.05 mg/mL to about 50 mg/mL, about 0.05 mg/mL to about 100 mg/mL, about 0.005 mg/mL to about 500 mg/mL, about 0.1 mg/mL to about 50 mg/mL, about 0.1 mg/mL to about 10 mg/mL, about 0.05 mg/mL to about 25 mg/mL, about 0.05 mg/mL to about 10 mg/mL, about 0.05 mg/mL to about 5 mg/mL, or about 0.05 mg/mL to about 1 mg/mL.

In some embodiments, the pharmaceutical composition includes a compound disclosed herein or a pharmaceutically acceptable salt thereof, or combination thereof, at a concentration of, e.g., about 0.05 mg/mL to about 15 mg/mL, about 0.5 mg/mL to about 10 mg/mL, about 0.25 mg/mL to about 5 mg/mL, about 0.5 mg/mL to about 7 mg/mL, about 1 mg/mL to about 10 mg/mL, about 5 mg/mL to about 10 mg/mL, about 5 mg/mL to about 15 mg/mL, about 5 mg/mL to 25 mg/mL, about 5 mg/mL to 50 mg/mL, or about 10 mg/mL to 100 mg/mL. In some embodiments, the pharmaceutical compositions are formulated as a total volume of about, e.g., 10 mL, 20 mL, 25 mL, 50 mL, 100 mL, 200 mL, 250 mL, or 500 mL.

Typically, dosages may be administered to a subject once, twice, three or four times daily, every other day, every three days, twice weekly, once weekly, twice monthly, or once monthly. In some embodiments, compound (i) and/or compound (ii) or combination thereof or a pharmaceutically acceptable salt thereof is administered to a subject once in the morning or once in the evening. In some embodiments, compound (i) and/or compound (ii) or combination thereof or a pharmaceutically acceptable salt thereof is administered to a subject once in the morning and once in the evening. In some embodiments, compound (i) and/or compound (ii) or combination thereof or a pharmaceutically acceptable salt thereof is administered to a subject three times a day (e.g., at breakfast, lunch, and dinner), at a dose, e.g., of about 50 mg/administration (e.g., 150 mg/day).

In some embodiments, compound (i) and/or compound (ii) or combination thereof or a pharmaceutically acceptable salt thereof is administered to a subject at a dose of 25 mg/day in one or more doses. In some embodiments, compound (i) and/or compound (ii) or combination thereof or a pharmaceutically acceptable salt thereof is administered to a subject at a dose of 50 mg/day in one or more doses. In some embodiments, compound (i) and/or compound (ii) or combination thereof or a pharmaceutically acceptable salt thereof is administered to a subject at a dose of 75 mg/day in one or more doses. In some embodiments, compound (i) and/or compound (ii) or combination thereof or a pharmaceutically acceptable salt thereof is administered to a subject at a dose of 100 mg/day in one or more doses. In some embodiments, compound (i) and/or compound (ii) or combination thereof or a pharmaceutically acceptable salt thereof is administered to a subject at a dose of 150 mg/day in one or more doses. In some embodiments, compound (i) and/or compound (ii) or combination thereof or a pharmaceutically acceptable salt thereof is administered to a subject at a dose of 200 mg/day in one or more doses. In some embodiments, compound (i) and/or compound (ii) or combination thereof or a pharmaceutically acceptable salt thereof is administered to a subject at a dose of 250 mg/day in one or more doses.

In some embodiments, the dosage of compound (i) and/or compound (ii) or combination thereof or a pharmaceutically acceptable salt thereof is 0.01-100 mg/kg, 0.5-50 mg/kg, 0.5-10 mg/kg or 25-50 mg/kg once, twice, three times or four times daily. For example, in some embodiments, the dosage of compound (i) and/or compound (ii) or combination thereof or a pharmaceutically acceptable salt thereof is 0.1 mg/kg, 0.25 mg/kg, 0.5 mg/kg, 1 mg/kg, 5 mg/kg, 7.5 mg/kg, or 10 mg/kg once, twice, three times or four times daily. In some embodiments, a subject is administered a total daily dose of 0.01 mg to 500 mg of compound (i) and/or compound (ii) or combination thereof or a pharmaceutically acceptable salt thereof once, twice, three times, or four times daily. In some embodiments, the total amount of compound (i) and/or compound (ii) or combination thereof or a pharmaceutically acceptable salt thereof administered to a subject in a 24-hour period is, e.g., 5 mg, 10 mg, 20 mg, 25 mg, 30 mg, 35 mg, 40 mg, 45 mg, 50 mg, 60 mg, 75 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, or 600 mg, or within a range bounded by any two of the foregoing values. In some embodiments, the subject may be started at a low dose and the dosage is escalated. In some embodiments, the subject may be started at a high dose and the dosage is decreased.

In some embodiments, compound (i) and/or compound (ii) or combination thereof or a pharmaceutically acceptable salt thereof may be administered at specified intervals. For example, during treatment, a patient may be administered compound (i) and/or compound (ii) or combination thereof or a pharmaceutically acceptable salt thereof at intervals of every, e.g., 1 year, 6 months, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

In some embodiments, compound (i) and/or compound (ii) or combination thereof or a pharmaceutically acceptable salt thereof is administered to a patient under the supervision of a healthcare provider. In some embodiments, compound (i) and/or compound (ii) or combination thereof or a pharmaceutically acceptable salt thereof is administered to a patient under the supervision of a healthcare provider at a clinic specializing in the delivery of psychoactive treatments. In some embodiments, compound (i) and/or compound (ii) or combination thereof or a pharmaceutically acceptable salt thereof is administered to a patient under the supervision of a healthcare provider at a dose intended to induce a psychedelic experience in the subject. In some embodiments, the administration to a patient under the supervision of a healthcare provider occurs periodically in order to maintain a therapeutic effect in the patient, e.g., every three days, twice weekly, once weekly, twice monthly, once monthly, thrice yearly, twice yearly, or once yearly. In some embodiments, compound (i) and/or compound (ii) or combination thereof or a pharmaceutically acceptable salt thereof is administered by a patient on their own at home or otherwise away from the supervision of a healthcare provider. In some embodiments, the administration by a patient on their own occurs periodically in order to maintain a therapeutic effect in the patient, e.g., daily, every other day, every three days, twice weekly, once weekly, twice monthly, or once monthly.

In one set of embodiments, the pharmaceutical composition containing components (i) and (ii) is admixed with or co-administered with one or more other therapeutic agents outside the scope of components (i) and (ii) and which may function to favorably modulate the activity of component (i) or (ii) or work independently in treating the mental disorder or condition and which do not adversely interact or interfere with the activity of component (i) or (ii). The co-administered therapeutic agent may be selected from any of the conventional therapeutic agents known in the art to be useful in treating a mental disorder or condition, e.g., an SSRI (e.g., citalopram, escitalopram, or fluoxetine) or non-SSRI (e.g. gabapentin or bupropion) for treating depression or anxiety. However, in some embodiments, one or more of any of the above co-administered therapeutic agents are excluded, or a co-administered therapeutic agent is altogether excluded, i.e., the pharmaceutical composition or treatment method may include or employ only components (i) and (ii), optionally in a pharmaceutically acceptable carrier. In a first instance, the co-administration may be accomplished by including components (i) and (ii) in admixture with one or more other therapeutic agents in the same pharmaceutical composition being administered. In a second instance, the co-administration may be accomplished by administering components (i) and (ii) separately from one or more other therapeutic agents, i.e., at the same time or at different times. In particular embodiments, a GABA receptor modulator or antagonist and/or a 5-HT2A receptor modulator agonist (e.g., a tryptamine, ergoline, phenethylamine, or amphetamine) or antagonist is included or excluded from the pharmaceutical composition or any of the treatment embodiments disclosed in the present disclosure.

In a first embodiment, therapeutic compounds (i) and (ii) are administered to the subject as a single pharmaceutical composition in which therapeutic compounds (i) and (ii) are present in pharmaceutically effective amounts. In a second embodiment, therapeutic compounds (i) and (ii) are administered to the subject in separate administrations within 7 days, 6 days, 5 days, 4 days, 3 days, or 48 hours of each other, wherein the term “within” indicates at (precisely or about) or under the specified time but with some minimal amount of time between administrations, e.g., at least 1, 2, 5, 10, 20, 30, or 60 minutes. In a third embodiment, therapeutic compounds (i) and (ii) are administered to the subject in separate administrations within 36 hours of each other. In a fourth embodiment, therapeutic compounds (i) and (ii) are administered to the subject in separate administrations within 24 hours of each other. In a fifth embodiment, therapeutic compounds (i) and (ii) are administered to the subject in separate administrations within 18 hours of each other. In a sixth embodiment, therapeutic compounds (i) and (ii) are administered to the subject in separate administrations within 12 hours of each other. In a seventh embodiment, therapeutic compounds (i) and (ii) are administered to the subject in separate administrations within 6 hours of each other. In an eighth embodiment, therapeutic compounds (i) and (ii) are administered to the subject in separate administrations within 4 hours of each other. In a ninth embodiment, therapeutic compounds (i) and (ii) are administered to the subject in separate administrations within 3 hours of each other. In a tenth embodiment, therapeutic compounds (i) and (ii) are administered to the subject in separate administrations within 2 hours of each other. In an eleventh embodiment, therapeutic compounds (i) and (ii) are administered to the subject in separate administrations within 1 hour of each other. In other embodiments, therapeutic compounds (i) and (ii) are administered in separate administrations within a range of time bounded by any two of the exemplary periods of time provided above (e.g., 1-48 hours, 2-48 hours, 1-24 hours, 2-24 hours, 1-12 hours, or 2-12 hours), or within a range of time having a minimum of 1, 2, 5, 10, 20, 30, or 60 minutes and any of the periods of time disclosed above (e.g., 30 minutes to 24 hours or 30 minutes to 12 hours). In some embodiments, therapeutic compound (i) is administered before therapeutic compound (ii). In other embodiments, therapeutic compound (ii) is administered before therapeutic compound (i).

In some embodiments, therapeutic compound (ii) is administered one or more times, such as by any of the dosages or dosing regimens described above, until a sufficient therapeutic response is achieved, and then therapeutic compound (i) is administered one or more times (by any of the dosages or dosing regimens described above) to extend or maintain that therapeutic response. In some embodiments, therapeutic compound (i) is administered daily or twice daily after a sufficient therapeutic response is achieved by administration of compound (ii). In other embodiments, therapeutic compounds (i) and (ii) are administered to the subject as a single pharmaceutical composition in which therapeutic compounds (i) and (ii) are present in pharmaceutically effective amounts, one or more times until a sufficient therapeutic response is achieved, and then therapeutic compound (i) is administered one or more times to extend or maintain that therapeutic response. For any of the foregoing embodiments, the therapeutic response may be a >50% (greater than 50%) reduction in score on the Hamilton Depression Rating Scale or Montgomery Asberg Depression Rating Scale.

In some embodiments, the therapeutic compounds (i) and (ii) are administered to the subject in amounts that are sub-therapeutic if administered alone, yet provide a synergistic effect that treats the mental disorder when administered in combination either as a single pharmaceutical composition or in separate administrations within 7 days, 6 days, 5 days, 4 days, 3 days, or 48 hours of each other or within any of the other time periods delineated earlier above. The sub-therapeutic dose varies according to the compound being administered and the mental condition or disorder being treated and can be independently selected for each compound from any of the doses disclosed anywhere within the present disclosure. The term “synergistic effect” indicates a positive effect in the treatment of the mental condition or disorder that exceeds the expected additive effect that each compound would be expected or known to provide if administered alone.

In some embodiments of the method, component (i) is a biarylpropylsulfonamide, which may be at least one of LY-451395 (mibampator), LY-404187, LY-503430, PF-04958242 (BIIB-104; pesampator), LY-392098, LY-450108, LY-451646, PF-04778574, CMPDA, CMPDB, and (R,R)-PIMSD, or more particularly, at least one of LY-451395 (mibampator), LY-404187, LY-503430, and PF-04958242 (BIIB-104; pesampator), or more particularly, PF-04958242 (BIIB-104; pesampator). In some embodiments, PF-04958242 is administered, and each dose of PF-04958242 is about 0.05 mg to about 1 mg or about 0.1 mg to about 0.5 mg.

In some embodiments of the method, component (ii) is selected from at least one of ketamine, R-ketamine, S-ketamine, S-norketamine, N-ethylnorketamine, N-propylnorketamine, memantine, amantadine, phencyclidine (PCP), rolicyclidine (PCPy), PCM, PCPr, tenocyclidine (TCP), tiletamine, 2-oxo-PCP, 2-oxo-PCE, gacyclidine, 3-MeO—PCP, 3-OH—PCP, 4-MeO—PCP, deschloroketamine, 2-fluoro-deschloroketamine, eticyclidine (PCE), 3-MeO—PCE, 4-MeO—PCE, 3-OH—PCE, methoxetamine, methoxmetamine, methoxpropamine, methoxisopropamine, and hydroxetamine. In more particular embodiments, component (ii) is selected from ketamine, R-ketamine, S-ketamine, and S-norketamine, or more particularly selected from ketamine, R-ketamine, and S-ketamine, or component (ii) may be or include ketamine or an analogue thereof.

In some embodiments of the method, component (ii) is selected from at least one of:

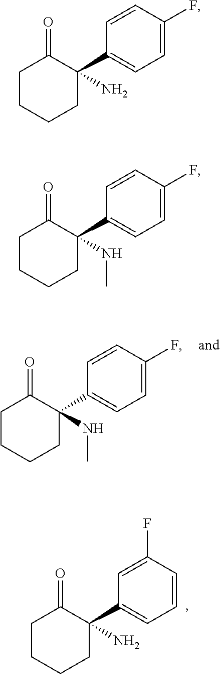 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]

or, more particularly, component (ii) is or includes

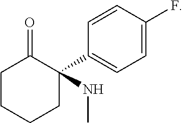 [SMILES: CN[C@@]1(c2ccc(F)cc2)CCCCC1=O]

In some embodiments of the method, component (ii) is selected from at least one of 8A-PDHQ, dextromethorphan, dextromethorphan-OD3, dextrorphan, dextrallorphan, dextromethadone (REL-1017), PD-137889, neramexane, memantine, NEFA, dizocilpine, lanicemine, diphenidine, ephenidine, isopropylphenidine, methoxphenidine, fluorolintane, xenon, krypton, nitrous oxide, etoxadrol, dexoxadrol, ibogaine, and noribogaine, or more particularly selected from dextromethorphan and dextromethorphan-OD3. In further embodiments, the subject may be administered a component (iii), which is a pharmaceutically effective amount of a third compound selected from quinidine and bupropion.

In some embodiments of the method, component (ii) is a compound that selectively blocks or negatively modulates NMDA receptors containing the GluN2B (GRIN2B) subunit. In more particular embodiments, component (ii) is selected from at least one of MIJ821, rislenemdaz, eliprodil, ifenprodil, besonprodil, traxoprodil, radiprodil, BMS-986169, BMS-986163, Ro 25-6981, Ro 04-5595, TCN 237, TCS 46b, and Co 101244.

In some embodiments of the method, a subject (as described earlier above) is administered a pharmaceutical composition containing the following components: (i) a positive allosteric modulator (PAM) of the AMPA receptor, or a pharmaceutically acceptable salt thereof; and (ii) (2R,3S)-2-amino-3-hydroxy-3-pyridin-4-yl-1-pyrrolidin-1-yl-propan-1-one, or a pharmaceutically acceptable salt thereof. Component (i) can be any of the compounds described throughout this disclosure for component (i). In some embodiments, component (i) is PF-04958242 (BIIB-104; pesampator). In some embodiments, each dose unit of the composition contains between about 0.05 and about 1 mg of PF-04958242 (BIIB-104; pesampator). In some embodiments, each dose unit of the composition contains between about 0.1 and about 0.5 mg of PF-04958242 (BIIB-104; pesampator). In further or separate embodiments, each dose unit of the composition contains between about 10 and about 100 mg of component (ii). In further or separate embodiments, each dose unit of the composition contains between about 20 and about 40 mg of component (ii). In some embodiments, the composition further includes a pharmaceutically acceptable carrier, as amply described earlier above. In some embodiments, therapeutic compounds (i) and (ii) are administered to the subject as a single pharmaceutical composition in which therapeutic compounds (i) and (ii) are present in pharmaceutically effective amounts. In other embodiments, therapeutic compounds (i) and (ii) are administered separately, such as within 7 days, 6 days, 5 days, 4 days, 3 days, or 48 hours of each other, as described earlier above. In some embodiments, therapeutic compound (i) is administered before therapeutic compound (ii). In other embodiments, therapeutic compound (ii) is administered before therapeutic compound (i).

The mental/psychiatric condition or disorder being treated can be any such condition or disorder known in the art, such as may be described in the DSM-5 manual. The mental/psychiatric condition or disorder may be, in general terms, for example, a form of depression, bipolar disorder, substance abuse disorder (e.g., drug addiction, or more particularly, opioid abuse disorder), anxiety, obsessive-compulsive and related disorders, trauma- and stressor-related disorders, feeding and eating disorders, borderline personality disorder, attention-deficit/hyperactivity disorders, autism spectrum disorders, neurocognitive disorders (e.g., Alzheimer's Disease, mild or moderate cognitive impairment, and Parkinson's Disease), or treatment-resistant disease or disorder. Any of the pharmaceutical compositions described above, including any of the combinations of compounds (i) and (ii) described above, in any of the dosages or dosage regimens described above can be used to treat any one or more of the mental/psychiatric conditions or disorders described in the present disclosure.

In a first set of embodiments, the mental/psychiatric condition or disorder being treated by use of any of the above described pharmaceutical compositions and dosages thereof is or includes a form of depression. The depressive condition or disorder may be, for example, major depressive disorder, persistent depressive disorder, postpartum depression, premenstrual dysphoric disorder, seasonal affective disorder, psychotic depression, disruptive mood dysregulation disorder, substance/medication-induced depressive disorder, and/or depressive disorder due to or related to another medical (possibly non-psychiatric) condition. The depression may also be a refractory depression, e.g., patients suffering from a depressive disorder that does not, and/or has not, responded to adequate courses of at least one, or at least two, other antidepressant compounds or therapeutics. As used herein, the term “depressive disorder” may include refractory depression.

In a second set of embodiments, the mental/psychiatric condition or disorder being treated by use of any of the above described pharmaceutical compositions and dosages thereof is or includes a form of bipolar disorder. The bipolar disorder may be, for example, bipolar disorder I, bipolar disorder II, cyclothymic disorder, substance/medication-induced bipolar and related disorder, and/or bipolar and related disorder due to another medical (possibly non-psychiatric) condition.

In a third set of embodiments, the mental/psychiatric condition or disorder being treated by use of any of the above described pharmaceutical compositions and dosages thereof is or includes a form of anxiety disorder. The anxiety disorder may be, for example, separation anxiety disorder, selective mutism, specific phobia, social anxiety disorder, panic disorder, panic attack, agoraphobia, generalized anxiety disorder, substance/medication-induced anxiety disorder, and/or anxiety disorder due to another medical (possibly non-psychiatric) condition.

In a fourth set of embodiments, the mental/psychiatric condition or disorder being treated by use of any of the above described pharmaceutical compositions and dosages thereof is or includes a form of substance abuse, substance addiction, or a related disorder. The substance may be any substance capable of being abused for achieving a psychoactive response, particularly substances that are addictive. The substance may be, for example, an opioid (e.g., heroin, oxycodone, or fentanyl), alcohol, cannabinoid (in isolated form or cannabis plant), inhalant, sedative, hypnotic, anxiolytic, stimulant (e.g., amphetamine or methamphetamine), nicotine (in isolated form or tobacco plant), and/or caffeine. In some embodiments, the method of treatment diminishes or prevents craving of a substance being abused and/or facilitates substance use cessation or withdrawal.

In a fifth set of embodiments, the mental/psychiatric condition or disorder being treated by use of any of the above described pharmaceutical compositions and dosages thereof is or includes a form of obsessive-compulsive (OCD) or related disorder. Some examples of OCD and related disorders include obsessive-compulsive disorder, body dysmorphic disorder, hoarding disorder, trichotillomania (hair-pulling disorder), excoriation (skin-picking) disorder, substance/medication-induced obsessive-compulsive and related disorder, and obsessive-compulsive and related disorder due to another medical condition.

In a sixth set of embodiments, the mental/psychiatric condition or disorder being treated by use of any of the above described pharmaceutical compositions and dosages thereof is or includes a form of trauma- and stressor-related disorders. Some examples of such disorders include reactive attachment disorder, disinhibited social engagement disorder, post-traumatic stress disorder, acute stress disorder, and adjustment disorders.

In a seventh set of embodiments, the mental/psychiatric condition or disorder being treated by use of any of the above described pharmaceutical compositions and dosages thereof is or includes a form of eating disorder. Some examples of eating disorders include anorexia nervosa, bulimia nervosa, binge-eating disorder, pica, rumination disorder, and avoidant/restrictive food intake disorder.

In an eighth set of embodiments, the mental/psychiatric condition or disorder being treated by use of any of the above described pharmaceutical compositions and dosages thereof is or includes a form of a neurocognitive disorder. Some examples of neurocognitive disorders include delirium, mild cognitive impairment, moderate cognitive impairment, major neurocognitive disorder, mild neurocognitive disorder, mild or advanced Alzheimer's disease, major or mild frontotemporal neurocognitive disorder, major or mild neurocognitive disorder with Lewy bodies, major or mild vascular neurocognitive disorder, major or mild neurocognitive disorder due to traumatic brain injury, substance/medication-induced major or mild neurocognitive disorder, major or mild neurocognitive disorder due to HIV infection, major or mild neurocognitive disorder due to prion disease, mild or advanced Parkinson's disease, mild or advanced Huntington's disease, major or mild neurocognitive disorder due to another medical condition, and major or mild neurocognitive disorder due to multiple etiologies.

In a ninth set of embodiments, the mental/psychiatric condition or disorder being treated by use of any of the above described pharmaceutical compositions and dosages thereof is or includes a neurodevelopmental disorder. Some examples of neurodevelopmental disorders include autism, autism spectrum disorder, attention-deficit/hyperactivity disorder, stereotypic movement disorder, tic disorders, Tourette's disorder, persistent (chronic) motor or vocal tic disorder, and provisional tic disorder.

In a tenth set of embodiments, the mental/psychiatric condition or disorder being treated by use of any of the above described pharmaceutical compositions and dosages thereof is or includes a personality disorder. Some examples of personality disorders include borderline personality disorder, paranoid personality disorder (e.g., paranoia), schizoid personality disorder (e.g., schizophrenia), schizotypal personality disorder, antisocial personality disorder, narcissistic personality disorder, and avoidant personality disorder.

In some embodiments, the method of treatment described above using any combination of compounds (i) and (ii) results in an improvement of any one or more of the mental conditions or disorders enumerated above. In some embodiments, the method may also provide an improvement in at least one symptom selected from any of the following: sadness, lethargy, or lassitude, depressed mood, inability to feel, anxious worried feelings, fears, feeling tense, feeling restlessness, diminished interest in all or nearly all activities, difficulty initiating activities, significant increased or decreased appetite leading to weight gain or weight loss, insomnia, irritability, fatigue, feelings of worthlessness or low self-esteem, strongly held negative beliefs or pessimistic thoughts about self, others or world, feelings of helplessness, inability to concentrate or distractibility, recurrent thoughts of death or suicide, feelings of guilt, memory complaints, difficulty experiencing positive feelings, feelings of being isolated from people, hypervigilance, risk taking behavior, avoidance of thoughts about a stressful or traumatic event, pains and aches, ruminations and obsessive thoughts, compulsive behaviors, disturbing intrusive thoughts, addictive drug use, guilt associated with drug use, and withdrawal symptoms due to drug use.

In some embodiments, the patient is treated with any of the above described pharmaceutical compositions and dosages thereof to result in one or more of the following improvements: (i) a 50% or greater improvement (relative to baseline) based on at least one assessment test selected from MADRS, HDRS, QIDS, HAM-D6, and adjusted HRDS17; (ii) 50% or greater improvement relative to the baseline in a score on the Ruminative Response Scale; (iii) 50% or greater improvement relative to the baseline in a score on the Snaith Hamilton Anhedonia Pleasure Scale; (iv) 50% or greater improvement relative to the baseline in a score on the Karolinska Sleepiness Scale; (v) 50% or greater improvement relative to the baseline in a score on the Digit Symbol Substitution Test; (vi) 50% or greater improvement relative to the baseline in a score on the Prolonged Grief Disorder-13; (vii) 50% or greater improvement in a HAM-A score relative to the baseline; (viii) 50% or greater improvement in a score on the Liebowitz Social Anxiety Scale relative to the baseline; (ix) 50% or greater improvement relative to the baseline in a score on the Yale-Brown Obsessive Compulsive Scale (YBOCS); (x) 50% or greater improvement relative to the baseline in a score on the Clinician-Administered PTSD Scale for DSM-5 (CAPS-5); or (xi) 50% or greater improvement relative to the baseline in a score on a scale selected from the group consisting of Conners' Adult ADHD Rating Scale-Investigator Rated, AAQOL-29, and cognitive testing (e.g., CogState or CANTAB).

Examples have been set forth below for the purpose of illustration and to describe the best mode of the invention at the present time. However, the scope of this invention is not to be in any way limited by the examples set forth herein.

### EXAMPLES

### Example 1. Studies of the Safety and Efficacy of Combinations Comprising an AMPAR-PAM and an NMDA Receptor Antagonist in Adults with a Neuropsychiatric Disorder

A study evaluating the safety and efficacy of a combination comprising an AMPAR-PAM compound and an NMDA receptor antagonist (NMDAR-ant) compound in adults with a psychiatric disorder is conducted. The subjects are diagnosed with a depressive disorder. Subjects initially receive assessment (e.g., MADRS, HDRS, QIDS, HAM-D6, and adjusted HRDS17) and behavioral therapy, such as prior day cognitive tasks or other behavioral training or therapy.

Following assessment and behavioral therapy, subjects are randomized into one of the following groups:
AMPAR-PAM compound+placebo
NMDAR-ant compound+placebo
AMPAR-PAM compound+NMDAR-ant compound

Subjects receive treatment with the combination or each individual component either 1-3 times per week for 2-6 weeks or daily for 2-6 weeks. Potentially less frequent maintenance therapy is used following response. The following combinations are evaluated:

An AMPAR-PAM compound (e.g., mibampator, LY-451395, BIIB-104, PF-04958242, tulrampator, CX-156, CS-1632, CX-691, or farampator) in combination with an NMDA receptor antagonist (e.g., Compound 18R, ketamine, R-ketamine, S-ketamine, N-ethylnorketamine, N-propylnorketamine, memantine, amantadine, phencyclidine (PCP), rolicyclidine (PCPy), PCM, PCPr, tenocyclidine (TCP), tiletamine, 2-oxo-PCP, 2-oxo-PCE, gacyclidine, 3-MeO—PCP, 3-OH—PCP, 4-MeO—PCP, deschloroketamine, 2-fluoro-deschloroketamine, eticyclidine (PCE), 3-MeO—PCE, 4-MeO—PCE, 3-OH—PCE, methoxetamine, methoxmetamine, methoxpropamine, methoxisopropamine, hydroxetamine, or 8A-PDHQ).

Following the above treatment, subjects are assessed based on at least one of the following assessment tests: MADRS, HDRS, QIDS, HAM-D6, and adjusted HRDS17. Some level of improvement is observed for all tested combinations, and at least a 50% improvement is found in at least some of the combinations tested. The combinations of AMPAR-PAM and NMDAR antagonist show superiority in durability, efficacy, and/or potency compared to the individual components in combination with placebo.

A randomized withdrawal design may also be employed for assessing maintenance:

Patients/subjects who are responsive to a combination (e.g., that have a 50% response to acute treatment) are blindly randomized to continue the treatment or to receive placebo. The patients/subjects are then monitored over time to assess the time to relapse or other negative outcome (e.g., a certain % worsening). The maintenance therapy with the combination provides better durability of response than placebo. Alternatively, subjects are first treated with one or more doses of an NMDAR antagonist (e.g., Compound 18R) and those subjects who are responsive to this treatment (e.g., that have a >50% response to acute treatment, e.g., reduction in MADRS score) are blindly randomized to receive daily or intermittent (e.g., 1-3 times per week) treatment with an AMPAR-PAM (e.g., BIIB-104 at a dose of, e.g., 0.1-0.5 mg per administration) or placebo. The patients/subjects are then monitored over time to assess the time to relapse or other negative outcome (e.g., a certain % worsening). The maintenance therapy with the AMPAR-PAM provides better durability of the initial response to the NMDAR antagonist than does placebo.

### Example 2. Activity of Combinations in the Rat Forced Swim Test

Disclosed combinations of AMPAR-PAMs (e.g., BIIB-104) and NMDAR-ants (e.g., compound 18R) induce antidepressant-like effects in the forced swim test (FST) in rats with a 23.5-h pre-treatment time. Specifically, the combined treatment with the two compounds reduces immobility time relative to vehicle control, indicative of an antidepressant-like effect. These effects on immobility are observed 23.5 hours after a single administration of the combination, a time point at which most or all of the drugs have been cleared from the systemic circulation, suggesting that the combination has both rapid-acting and long-lasting antidepressant-like effects. Further, the minimally effective doses of each component when administered together as a combination treatment are lower than when each compound is administered alone. In other words, the components of the combination potentiate each other to provide a more potent antidepressant-like effect in this rat model.

Animals. Male Sprague Dawley rats, aged 8-10 weeks, are used in the experiments. Animals are housed in groups of 2 under controlled temperature (22±3° C.) and relative humidity (30-70%) conditions, with 12-hour light/dark cycles, and with ad libitum food and water. All efforts are made to minimize suffering.

Drugs and Drug Administration. Test compounds, vehicle, and the positive control desipramine are administered subcutaneously (s.c.), with doses calculated based on the free base. A pharmacologically inert vehicle is used. All compounds are administered at a volume of 5 mL/kg. Test compounds (in each case, an AMPAR-PAM+an NMDAR-ant together, or either compound alone) and vehicle are administered 0.5 h after the start of the training swim (Swim 1) and 23.5 h before the test swim (Swim 2). Desipramine is administered 3 times, at 23.5 h, 5 h, and 1 h before the test swim (Swim 2), each time at a dose of 20 mg/kg.

Forced Swim Test (FST). Animals are randomized based on body weight, and it is ensured that inter-group variations are minimal and do not exceed ±20% of the mean body weight across the groups. Group size is N=10 per treatment. Rats are handled for about 2 min daily for the 5 days prior to the beginning of the experimental procedure. On the first day of the experiment (i.e., Day 0), post randomization, training swim sessions (Swim 1) are conducted between 12:00 and 18:00 h with all animals by placing rats in individual glass cylinders (46 cm tall×20 cm in diameter) containing 23-25° C. water 30 cm deep for 15 minutes. At the conclusion of Swim 1, animals are dried with paper towels, placed in heated drying cages for 15 minutes, and then returned to their home cages. Animals are then administered the appropriate drug or vehicle treatment(s), as described above. For clarity, a compound administration time of 23.5 h before Swim 2 means 0.5 h after the start of Swim 1 and 0.25 h after the completion of Swim 1 (i.e., immediately after return to the home cage). On Day 1 (i.e., 24 h after start of Swim 1), animals perform the test swim (Swim 2) for a period of 5 min but otherwise under the same conditions as Swim 1. During all swim sessions, the water is changed between each animal.

Behavioral scoring is conducted by observers who are blind to the treatment groups. Animals are continuously observed during Swim 2 and the total time spent engaging in the following behaviors is recorded: immobile, swimming, and climbing. A rat is judged to be immobile when it remains floating in the water without struggling and is making only those movements necessary to keep its head above water. A rat is judged to be swimming when it makes active swimming motions, more than necessary to merely maintain its head above water (e.g., moving around in the cylinder). A rat is judged to be climbing when it makes active movements with its forepaws in and out of the water, usually directed against the walls.

Statistical Analysis. Analysis is performed using GraphPad Prism 9. Comparisons between groups are performed using the one-way analysis of variance (ANOVA), followed by Dunnett's test for comparisons to vehicle.

### Example 3. Activity of a BIIB-104/Compound 18R Combination in the Rat Forced Swim Test

Animals. Male Sprague Dawley rats, aged 8-10 weeks, are used in the experiments. Animals are housed in groups of 2 under controlled temperature (22±3C) and relative humidity (30-70%) conditions, with 12-hour light/dark cycles, and with ad libitum food and water. All efforts are made to minimize suffering.

Drugs and Drug Administration. Test compounds, vehicle, and the positive control desipramine are administered subcutaneously (s.c.), with doses calculated based on the free base. A pharmacologically inert vehicle is used. All compounds are administered at a volume of 5 mL/kg. Test compounds or vehicle are administered 0.5 h after the start of the training swim (Swim 1) and 23.5 h before the test swim (Swim 2), as described in more detail below. Desipramine is administered 3 times, at 23.5 h, 5 h, and 1 h before the test swim (Swim 2), each time at a dose of 20 mg/kg.

Forced Swim Test (FST). Animals are randomized based on body weight, and it is ensured that inter-group variations are minimal and do not exceed ±20% of the mean body weight across the groups. Group size is N=10 per treatment. Rats are handled for about 2 min daily for the 5 days prior to the beginning of the experimental procedure. On the first day of the experiment (i.e., Day 0), post randomization, training swim sessions (Swim 1) are conducted between 12:00 and 18:00 h with all animals by placing rats in individual glass cylinders (46 cm tall×20 cm in diameter) containing 23-25° C. water 30 cm deep for 15 minutes. At the conclusion of Swim 1, animals are dried with paper towels, placed in heated drying cages for 15 minutes, and then returned to their home cages. Animals are then administered vehicle, desipramine (1st dose), or Compound 18R (1, 3.2 or 10 mg/kg) followed by a second injection of vehicle or BIIB-104 (0.03 mg/kg) within 10 minutes (desipramine animals receive a vehicle injection). For clarity, a compound administration time of 23.5 h before Swim 2 means 0.5 h after the start of Swim 1 and 0.25 h after the completion of Swim 1 (i.e., immediately after return to the home cage). On Day 1 (i.e., 24 h after start of Swim 1), animals perform the test swim (Swim 2) for a period of 5 min but otherwise under the same conditions as Swim 1. During all swim sessions, the water is changed between each animal.

Behavioral scoring is conducted by observers who are blind to the treatment groups. Animals are continuously observed during Swim 2 and the total time spent engaging in the following behaviors is recorded: immobile, swimming, and climbing. A rat is judged to be immobile when it remains floating in the water without struggling and is making only those movements necessary to keep its head above water. A rat is judged to be swimming when it makes active swimming motions, more than necessary to merely maintain its head above water (e.g., moving around in the cylinder). A rat is judged to be climbing when it makes active movements with its forepaws in and out of the water, usually directed against the walls.

Statistical Analysis. Analysis is performed using GraphPad Prism 9. Comparisons between groups are performed using the one-way analysis of variance (ANOVA), followed by Dunnett's test for comparisons to vehicle.

Results. Compound 18R produces a dose-dependent decrease in immobility time, indicating an antidepressant-like effect, and this effect is potentiated (i.e., there is a reduction of the minimal dose required to reduce immobility) by co-administration of BIIB-104.

### Example 4. Chronic Mild Stress

Animals. Male Wistar Han or Wistar Kyoto rats (Charles River, Sulzfeld, Germany) are brought into the laboratory at the age of 5 weeks (around 100 g), approximately one month before the start of the experiment. Except for the first 10 days after arrival, when the animals are housed in groups of 10, they are singly housed with food and water freely available, and maintained on a 12-h light/dark cycle in constant temperature (22±2° C.) and humidity (45±5%) conditions.

Drugs and Drug Administration. Test compounds or vehicle are administered subcutaneously (s.c.), with doses calculated based on the free base. A pharmacologically inert vehicle (e.g., saline) is used. All compounds are administered at a volume of 2 mL/kg. Test compounds (in each case, BIIB-104+Compound 18R together, or either compound alone) and vehicle are administered as described below.

Chronic Mild Stress (CMS) Procedure and Sucrose Preference. Male rats (250-350 g) are habituated to drinking 1% sucrose to establish a stable level of sucrose intake. Rats are assigned to either the CMS or the control (non-stressed) paradigm such that sucrose intake is balanced across the 2 cohorts. The CMS procedure is applied for a period of up to 10 consecutive weeks. Each week of the stress regime consists of two periods of food or water deprivation, two periods of 45-degree cage tilt, two periods of intermittent illumination (light on and off every 2 h), two periods of soiled cage (250 mL water in sawdust bedding), one period of paired housing, two periods of low intensity stroboscopic illumination (150 flashes/min), and three periods of no stress. All the stressors are 10-14 h in duration and are applied individually and continuously, day and night. Control, non-stressed animals are housed in separate rooms and have no contact with the stressed animals. Control rats are deprived of food and water for 14 h before each sucrose test, but otherwise food and water are available at libitum. After 2 weeks of stress or control paradigm, rats are assigned to one of 4 treatment groups (n=8/group) with equivalent baseline sucrose intake in all groups. Rats continue to be exposed to the stress paradigm for at least 4 weeks.

As shown in Table 1, rats are administered Compound 18R (1.5 mg/kg, i.p.), BIIB-104 (0.03 mg/kg, s.c.), or vehicle and 24 h later, sucrose intake is measured. After sucrose intake has been measured, rats are dosed with BIIB-104 or vehicle once daily for up to 4 weeks (Table 1).

TABLE 1
Drug administration schedule for CMS experiment.

| | Day 1 | Days 2-28 |
|---|---|---|
| Control C1 | Vehicle | Vehicle |
| Control C2 | Compound 18R | Vehicle |
| Control C3 | BIIB-104 | BIIB-104 |
| Control C4 | Compound 18R | BIIB-104 |
| Stress S1 | Vehicle | Vehicle |
| Stress S2 | Compound 18R | Vehicle |
| Stress S3 | BIIB-104 | BIIB-104 |
| Stress S4 | Compound 18R | BIIB-104 |

Sucrose intake is measured once per week, in each case before drug administration for that day (˜24 h after the previous drug administration). Administration of Compound 18R produces an increase in sucrose intake in stressed rats (significant increase vs vehicle-treated stressed rats) that is apparent 24 h later and is sustained for 1-2 weeks. Administration of BIIB-104 from day 2 onwards extends the durability of the effect of Compound 18R (S4) on sucrose intake, with sucrose intake remaining significantly higher than in vehicle-treated stressed rats longer than in the group dosed with Compound 18R followed by daily vehicle injections (S2), e.g., through weeks 3 and 4 of dosing. None of the drug treatments alters behavior in unstressed rats.

Elevated Plus Maze (EPM), Animals are tested in two non-transparent boxes, which consist of two open (50×11×40 cm) and two closed (50×11×40 cm) arms. The apparatus is elevated 50 cm above the floor and illuminated by two 25 W bulbs located beneath the open arms. The animals are placed in the center of the apparatus and the time spent in each arm, and number of entries into open and closed arms are recorded during a 5-min test.

All rats (all groups in Table 1) are assessed in the EPM in the third week of drug testing (typically day 16). Stressed vehicle-treated rats (S1) have higher levels of anxiety measured by a lower % time spent in open arms compared to control vehicle-treated rats (C1). Stressed Compound 18R-treated rats (S2) spend greater time in the open arms than stressed vehicle-treated rats (S1), but less time than unstressed vehicle-treated rats (C1) as the test is run 2 weeks after a single dose of Compound 18R. The combined treatment of Compound 18R followed by BIIB-104 fully reverses the effect of stress such that group S4 shows time spent in the open arms no different from group C1 and significantly greater than group S1. This indicates that BIIB-104 prolongs the efficacy of Compound 18R in reversing the anxiogenic effects of stress. None of the drug treatments alters behavior in unstressed rats.

Novel Object Recognition (NOR) Test. Animals are tested in an opaque circular open field (100 cm in diameter, 35 cm high, floor divided into painted 16-cm squares). After a period of 2 days adaptation to the open field (10 min daily), the animals are allowed to explore two identical cylinder-shaped white objects (7 cm in diameter, 11 cm high) for the time required to complete 15 s of exploration of both objects (T1 session). In the retention trial (T2 session) conducted one hour later, one of the objects presented previously is replaced by a novel prism-shaped black object (5 cm wide, 14 cm high). Rats are returned to the open field and the duration of exploration of each object is recorded in a 5-min test. A recognition index is calculated according to the formula: time of novel object exploration minus time of familiar object exploration, divided by total exploration time (novel plus familiar objects). During NOR sessions, the number of line crossings is recorded as a measure of locomotor activity.

All rats (all groups in Table 1) are assessed in the NOR in the third week of drug testing (typically day 17). The stressed vehicle-treated rats (S1) have impaired recognition memory indicated by a lower recognition index than control rats dosed with vehicle (C1), meaning S1 rats spend equal time exploring the novel and the familiar object, while C1 rats spend more time exploring the novel object. Stressed Compound 18R-treated rats (S2) show limited preference for exploring the novel object as the test is being run 2 weeks after a single dose of Compound 18R. The combined treatment of Compound 18R followed by BIIB-104 fully reverses the effect of stress such that group S4 shows a preference for the novel object no different from group C1 and significantly greater than group S1. This indicates that BIIB-104 prolongs the efficacy of Compound 18R in reversing the recognition memory-impairing effects of stress. None of the drug treatments alters behavior in unstressed rats.

### Example 5. Rotarod

Animals. Male C57BL/6 mice (8-10 weeks old) are housed in groups of >2 under standard environmentally controlled conditions (22 to 24° C.; relative humidity of 30 to 70%) with a standard 12-h light cycle. Food and water are available ad libitum.

Drugs and Drug Administration. Test compounds and vehicle are administered subcutaneously (s.c.), with doses calculated based on the free base. A pharmacologically inert vehicle is used. All compounds are administered at a volume of 10 mL/kg. Test compounds (in each case, BIIB-104+Compound 18R together, or either compound alone) and vehicle are administered as described below.

Rotarod Procedure. Mice will be trained to stay on a rotating bar (‘rotarod’) on days −4 and −2 by being placed on a bar rotating at 4 rpm for 3 min for 3 sessions separated by ˜10 min. On days −1 and 0, this will be repeated using a faster speed of 15 rpm. Mice that have a fall latency <80 s during training will be omitted from the study. On day 1, mice will be administered (s.c.) vehicle or BIIB-104 (0.03 mg/kg) 0-30 min prior to vehicle or Compound 18R (3.2, 10, or 32 mg/kg), for a total of 8 groups of n=10-12/group. Five min after the second injection, mice will be placed on the rotarod which will accelerate from 4 to 40 rpm over 300 s. The latency of mice to fall will be recorded. Mice will be returned to a standard cage until 30 min after the second dose, at which point the test on the accelerating rotarod will be repeated.

Results. Compound 18R dose-dependently reduces the latency of mice to fall off the rotarod with an ED50 of ˜20 mg/kg at both time points. BIIB-104 alone does not change latency to fall at either time point. Pretreatment/co-administration of BIIB-104 attenuates the reduction in latency to fall caused by Compound 18R alone and results in a higher ED50 at both time points. This indicates that BIIB-104 can reduce motor impairment caused by Compound 18R.

### Example 6. Visual Discrimination

Animals Male C57BL/6 mice (8-10 weeks old) are housed in groups of >2 under standard environmentally controlled conditions (22 to 24° C.; relative humidity of 30 to 70%) with a standard 12-h light cycle. Five days prior to the start of operant conditioning experiments, all subjects are placed on a food restriction diet designed to maintain a stable 80-85% of free feeding weight.

Drugs and Drug Administration. Test compounds and vehicle are administered subcutaneously (s.c.), with doses calculated based on the free base. A pharmacologically inert vehicle is used. All compounds are administered at a volume of 10 mL/kg. Test compounds (in each case, BIIB-104+Compound 18R together, or either compound alone) and vehicle are administered as described below. Mice are tested in a randomized cross-over design such that different compound combinations are tested in the same mice and a sample size of n=8-12/group is achieved. Compound testing sessions are separated by at least 3 days.

Visual Discrimination Procedure. A cohort of mice is trained to perform the visual discrimination task according to published methods (Dillon et al., 2009, Behavioural Brain Research 204, 67-76). The testing apparatus is a standard operant chamber (Med Associates, St Albans, VT). Food restricted mice are trained to press levers according to a stimulus light and are required to discriminate between reinforced and non-reinforced levers. Mice are required to press the lever signaled by the light stimulus in order to obtain a food reward. Mice are trained daily for ˜10 days to reach a high level (˜80%) of performance accuracy. Once training is complete, mice are used to assess the effects of test compounds. Part 1: mice are administered (s.c.) vehicle or Compound 18R (10, 32, or 100 mg/kg) 0-30 min prior to performing the visual discrimination task to identify a dose of Compound 18R that causes a reduction is task accuracy. The dose of Compound 18R that causes a reduction in accuracy is then tested in Part 2 in combination with BIIB-104. Part 2: Mice are dosed (s.c.) with BIIB-104 (0.03 mg/kg) or vehicle 0-30 min prior to vehicle or Compound 18R (dose identified in Part 1). Zero to 30 min after the second injection, mice are placed in the operant chamber and visual discrimination testing is performed.

Results. Compound 18R dose-dependently decreases visual discrimination test accuracy. Pretreatment/co-administration of BIIB-104 attenuates the reduction in test accuracy caused by Compound 18R alone. This indicates that BIIB-104 can reduce cognitive impairment caused by Compound 18R.

While there have been shown and described what are at present considered the preferred embodiments of the invention, those skilled in the art may make various changes and modifications which remain within the scope of the invention defined by the appended claims.

What is claimed is:

1. A pharmaceutical composition comprising the following components:
(i) a positive allosteric modulator (PAM) of the AMPA receptor; and
(ii) an NMDA receptor antagonist.

2. The composition of claim 1, wherein component (i) is an ampakine.

3. The composition of claim 2, wherein the ampakine is selected from the group consisting of CX-516 (Ampalex), CX-546, CX-554, CX-614, CX-691 (farampator), CX-717, CX-1632 (tulrampator), CX-1739, ORG-26576, ORG-2448, CX-701, CX-929, CX-1501, CX-1796, CX-1837, CX-1846, CX-1942, CX-2007, CX-2076, and S-70340.

4. The composition of claim 2, wherein the ampakine is selected from the group consisting of CX-1632, ORG-26576, CX-516, and CX-691.

5. The composition of claim 1, wherein component (i) is a biarylpropylsulfonamide.

6. The composition of claim 5, wherein the biarylpropylsulfonamide is selected from the group consisting of LY-451395 (mibampator), LY-404187, LY-503430, PF-04958242 (BIIB-104; pesampator), LY-392098, LY-450108, LY-451646, PF-04778574, CMPDA, CMPDB, and (R,R)-PIMSD.

7. The composition of claim 5, wherein the biarylpropylsulfonamide is selected from the group consisting of LY-451395 (mibampator), LY-404187, LY-503430, and PF-04958242 (BIIB-104; pesampator).

8. The composition of claim 5, wherein the biarylpropylsulfonamide is PF-04958242 (BIIB-104; pesampator).

9. The composition of claim 8, wherein each dose unit of the composition contains between about 0.05 and about 1 mg of PF-04958242 (BIIB-104; pesampator).

10. The composition of claim 9, wherein each dose unit of the composition contains between about 0.1 and about 0.5 mg of PF-04958242 (BIIB-104; pesampator).

11. The composition of claim 1, wherein component (i) is a benzothiadiazide.

12. The composition of claim 11, wherein the benzothiadiazide is selected from the group consisting of BIIR-777, cyclothiazide, diazoxide, hydrochlorothiazide (HCTZ), IDRA-21, and S-18986.

13. The composition of claim 1, wherein component (i) is a racetam.

14. The composition of claim 13, wherein the racetam is selected from the group consisting of piracetam, aniracetam, phenylpiracetam, oxiracetam, pramiracetam, seletracetam, levetiracetam, coluracetam, fasoracetam, brivaracetam, methylphenylpiracetam, dimiracetam, nebracetam, rolziracetam, and nefiracetam.

15. The composition of claim 13, wherein the racetam is selected from the group consisting of piracetam and aniracetam.

16. The composition of claim 1, wherein component (i) is selected from the group consisting of PEPA, PF-04701475, GVS-111, and TAK-653.

17. The composition of any one of claims 1-16, wherein component (ii) is an arylcyclohexylamine.

18. The composition of claim 17, wherein the arylcyclohexylamine has the following structure: 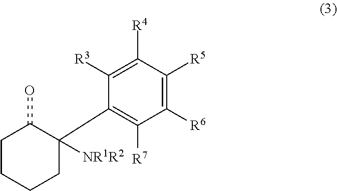
wherein:
the dashed double bond in Formula (3) indicates the optional presence of a keto group;
R¹ and R² are independently selected from H and R^{a}, wherein R^{a} is independently selected from H, C₁₋₁₀ alkyl, C₂₋₁₀ alkenyl, C₂₋₁₀ alkynyl, and C₃₋₇ cycloalkyl, and wherein R^{a} optionally contains one or more heteroatoms selected from O, N, S, and halogens, and wherein R¹ and R² are optionally interconnected to form an N-containing ring;
R³, R⁴, R⁵, R⁶, and R⁷ are independently selected from H, halogen, R^{b}, —CF₃, —OR^{b}, —C(O)R^{b}, —C(O)O(R^{b}), —C(O)NR^{b}₂, —OC(O)R^{b}, —NR^{b}₂, —NR^{b}C(O)R^{b}, —NR^{b}C(O)NR^{b}—, —NO₂, —CN, —OP(═O)O₂R^{b}₂, —SR^{b}, —S(O)R^{b}, —SO₂R^{b}, and —SO₂NR^{b}₂, wherein R^{b} is independently selected from H, C₁₋₁₀ alkyl, C₂₋₁₀ alkenyl, C₂₋₁₀ alkynyl, C₃₋₇ cycloalkyl, aryl, and heteroaryl, any of which is optionally substituted with halogen atoms;
and pharmaceutically acceptable salts thereof.

19. The composition of claim 18, wherein component (ii) is selected from the group consisting of ketamine, R-ketamine, S-ketamine, S-norketamine, N-ethylnorketamine, N-propylnorketamine, memantine, amantadine, phencyclidine (PCP), rolicyclidine (PCPy), PCM, PCPr, tenocyclidine (TCP), tiletamine, 2-oxo-PCP, 2-oxo-PCE, gacyclidine, 3-MeO—PCP, 3-OH—PCP, 4-MeO—PCP, deschloroketamine, 2-fluoro-deschloroketamine, eticyclidine (PCE), 3-MeO—PCE, 4-MeO—PCE, 3-OH—PCE, methoxetamine, methoxmetamine, methoxpropamine, methoxisopropamine, and hydroxetamine.

20. The composition of claim 18, wherein component (ii) is selected from the group consisting of:

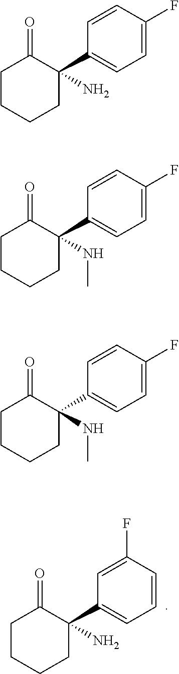 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]

21. The composition of claim 18, wherein component (ii) is selected from the group consisting of ketamine, R-ketamine, S-ketamine, and S-norketamine.

22. The composition of claim 18, wherein component (ii) is:

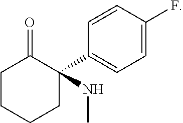 [SMILES: CN[C@@]1(c2ccc(F)cc2)CCCCC1=O]

23. The composition of claim 18, wherein component (ii) is selected from the group consisting of ketamine, R-ketamine, and S-ketamine.

24. The composition of claim 17, wherein component (ii) is ketamine or an analogue thereof.

25. The composition of any one of claims 1-16, wherein component (ii) is selected from the group consisting of 8A-PDHQ, dextromethorphan, dextromethorphan-OD3, dextrorphan, dextrallorphan, dextromethadone (REL-1017), PD-137889, neramexane, memantine, NEFA, dizocilpine, lanicemine, diphenidine, ephenidine, isopropylphenidine, methoxphenidine, fluorolintane, xenon, krypton, nitrous oxide, etoxadrol, dexoxadrol, ibogaine, and noribogaine.

26. The composition of claim 25, wherein component (ii) is selected from the group consisting of dextromethorphan or dextromethorphan-OD3.

27. The composition of claim 26, further comprising: (iii) a third compound selected from the group consisting of quinidine and bupropion.

28. The composition of any one of claims 1-16, wherein component (ii) is a compound that selectively blocks or negatively modulates NMDA receptors containing the GluN2B (GRIN2B) subunit.

29. The composition of claim 28, wherein component (ii) is selected from the group consisting of MIJ821, rislenemdaz, eliprodil, ifenprodil, besonprodil, traxoprodil, radiprodil, BMS-986169, BMS-986163, Ro 25-6981, Ro 04-5595, TCN 237, TCS 46b, and Co 101244.

30. The composition of any one of claims 1-29, further comprising a pharmaceutically acceptable carrier.

31. A method for treating a mental disorder, the method comprising administering to a subject having said mental disorder pharmaceutically effective amounts of the following therapeutic compounds:
(i) a positive allosteric modulator (PAM) of the AMPA receptor; and
(ii) an NMDA receptor antagonist.

32. The method of claim 31, wherein therapeutic compounds (i) and (ii) are administered to the subject as a single pharmaceutical composition in which therapeutic compounds (i) and (ii) are present in pharmaceutically effective amounts.

33. The method of claim 31, wherein therapeutic compounds (i) and (ii) are administered to the subject in separate administrations within 7 days of each other.

34. The method of claim 31, wherein therapeutic compounds (i) and (ii) are administered to the subject in separate administrations within 48 hours of each other.

35. The method of claim 31, wherein therapeutic compounds (i) and (ii) are administered to the subject in separate administrations within 24 hours of each other.

36. The method of claim 31, wherein therapeutic compounds (i) and (ii) are administered to the subject in separate administrations within 12 hours of each other.

37. The method of claim 31, wherein therapeutic compounds (i) and (ii) are administered to the subject in separate administrations within 6 hours of each other.

38. The method of claim 31, wherein therapeutic compound (i) is administered before therapeutic compound (ii).

39. The method of claim 31, wherein therapeutic compound (ii) is administered before therapeutic compound (i).

40. The method of claim 39, wherein therapeutic compound (ii) is administered one or more times until a sufficient therapeutic response is achieved and then therapeutic compound (i) is administered one or more times to extend or maintain that therapeutic response.

41. The method of claim 40, wherein the therapeutic response is a >50% reduction in score on the Hamilton Depression Rating Scale or Montgomery Asberg Depression Rating Scale.

42. The method of claim 40, wherein the therapeutic compound (i) is administered daily or twice daily.

43. The method of claim 31, wherein therapeutic compounds (i) and (ii) are administered to the subject as a single pharmaceutical composition in which therapeutic compounds (i) and (ii) are present in pharmaceutically effective amounts, one or more times until a sufficient therapeutic response is achieved, and then therapeutic compound (i) is administered one or more times to extend or maintain that therapeutic response.

44. The method of claim 31, wherein therapeutic compounds (i) and (ii) are administered to the subject in amounts that are sub-therapeutic if administered alone, yet provide a synergistic effect that treats the mental disorder when administered in combination, either as a single pharmaceutical composition or in separate administrations within 7 days of each other.

45. The method of claim 31, wherein the mental disorder is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, and depressive disorder due to another medical condition.

46. The method of claim 31, wherein the mental disorder is selected from the group consisting of bipolar disorder I, bipolar disorder II, cyclothymic disorder, substance/medication-induced bipolar and related disorder, and bipolar and related disorder due to another medical condition.

47. The method of claim 31, wherein the mental disorder is a substance-related or substance use disorder.

48. The method of claim 31, wherein the mental disorder is selected from the group consisting of separation anxiety disorder, selective mutism, specific phobia, social anxiety disorder, panic disorder, panic attach, agoraphobia, generalized anxiety disorder, substance/medication-induced anxiety disorder, anxiety disorder due to another medical condition.

49. The method of claim 31, wherein the mental disorder is selected from the group consisting of obsessive-compulsive and related disorders, trauma- and stressor-related disorders, feeding and eating disorders, borderline personality disorder, attention-deficit/hyperactivity disorder, and autism spectrum disorder.

50. The method of claim 31, wherein the mental disorder is a neurocognitive disorder.

51. The method of claim 31, wherein the mental disorder is a treatment-resistant disease or disorder.

52. The method of claim 31, wherein the method provides improvement in at least one symptom selected from the group consisting of sadness or lethargy or lassitude, depressed mood, inability to feel, anxious worried feelings, fears, feeling tense, feeling restlessness, diminished interest in all or nearly all activities, difficulty initiating activities, significant increased or decreased appetite leading to weight gain or weight loss, insomnia, irritability, fatigue, feelings of worthlessness or low self-esteem, strongly held negative beliefs or pessimistic thoughts about self, others or world, feelings of helplessness, inability to concentrate or distractibility, recurrent thoughts of death or suicide, feelings of guilt, memory complaints, difficulty experiencing positive feelings, feeling cut off or distant from people, hypervigilance, risk taking behavior, avoidance of thoughts about a stressful or traumatic event, pains and aches, ruminations and obsessive thoughts, compulsive behaviors, talking to people you don't know well or strangers, being center of attention, disturbing intrusive thoughts, can't get through week without drug use, guilty about drug use, problems with friends or family due to drug use, and withdrawal symptoms due to drug use.

53. The method of any one of claims 31-52, wherein component (i) is an ampakine.

54. The method according to claim 53, wherein the ampakine is selected from the group consisting of CX-516 (Ampalex), CX-546, CX-554, CX-614, CX-691 (farampator), CX-717, CX-1632 (tulrampator), CX-1739, ORG-26576, ORG-24448, CX-701, CX-929, CX-1501, CX-1796, CX-1837, CX-1846, CX-1942, CX-2007, CX-2076, and S-70340.

55. The method of claim 54, wherein the ampakine is selected from the group consisting of CX-1632, ORG-26576, CX-516, and CX-691.

56. The method of any one of claims 31-52, wherein component (i) is a biarylpropylsulfonamide.

57. The method of claim 56, wherein the biarylpropylsulfonamide is selected from the group consisting of LY-451395 (mibampator), LY-404187, LY-503430, PF-04958242 (BIIB-104; pesampator), LY-392098, LY-450108, LY-451646, PF-04778574, CMPDA, CMPDB, and (R,R)-PIMSD.

58. The method of claim 57, wherein the biarylpropylsulfonamide is selected from the group consisting of LY-451395 (mibampator), LY-404187, LY-503430, and PF-04958242 (BIIB-104; pesampator).

59. The method of claim 58, wherein the biarylpropylsulfonamide is PF-04958242 (BIIB-104; pesampator).

60. The method of claim 59, wherein each dose of PF-04958242 (BIIB-104; pesampator) is about 0.05 mg to about 1 mg.

61. The method of claim 60, wherein each dose of PF-04958242 (BIIB-104; pesampator) is about 0.1 mg to about 0.5 mg.

62. The method of any one of claims 31-52, wherein component (i) is a benzothiadiazide.

63. The method of claim 62, wherein the benzothiadiazide is selected from the group consisting of BIIR-777, cyclothiazide, diazoxide, hydrochlorothiazide (HCTZ), IDRA-21, and S-18986.

64. The method of any one of claims 31-52, wherein component (i) is a racetam.

65. The method of claim 64, wherein the racetam is selected from the group consisting of piracetam, aniracetam, phenylpiracetam, oxiracetam, pramiracetam, seletracetam, levetiracetam, coluracetam, fasoracetam, brivaracetam, methylphenylpiracetam, dimiracetam, nebracetam, rolziracetam, and nefiracetam.

66. The method of claim 65, wherein the racetam is selected from the group consisting of piracetam and aniracetam.

67. The method of any one of claims 31-52, wherein component (i) is selected from the group consisting of PEPA, PF-04701475, GVS-111, and TAK-653.

68. The method of any one of claims 31-67, wherein component (ii) is an arylcyclohexylamine.

69. The method of claim 68, wherein the arylcyclohexylamine has the following structure: 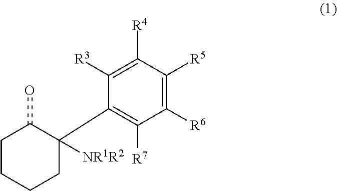
wherein:
the dashed double bond in Formula (3) indicates the optional presence of a keto group;
R¹ and R² are independently selected from H and R^{a}, wherein R^{a} is independently selected from H, C₁₋₁₀ alkyl, C₂₋₁₀ alkenyl, C₂₋₁₀ alkynyl, and C₃₋₇ cycloalkyl, and wherein R^{a} optionally contains one or more heteroatoms selected from O, N, S, and halogens, and wherein R¹ and R² are optionally interconnected to form an N-containing ring;
R³, R⁴, R⁵, R⁶, and R⁷ are independently selected from H, halogen, R^{b}, —CF₃, —OR^{b}, —C(O)R^{b}, —C(O)O(R^{b}), —C(O)NR^{b}₂, —OC(O)R^{b}, —NR^{b}₂, —NR^{b}C(O)R^{b}, —NR^{b}C(O)NR^{b}—, —NO₂, —CN, —OP(═O)O₂R^{b}₂, —SR^{b}, —S(O)R^{b}, —SO₂R^{b}, and —SO₂NR^{b}₂, wherein R^{b} is independently selected from H, C₁₋₁₀ alkyl, C₂₋₁₀ alkenyl, C₂₋₁₀ alkynyl, C₃₋₇ cycloalkyl, aryl, and heteroaryl, any of which is optionally substituted with halogen atoms;
and pharmaceutically acceptable salts thereof.

70. The method of claim 69, wherein component (ii) is selected from the group consisting of ketamine, R-ketamine, S-ketamine, S-norketamine, N-ethylnorketamine, N-propylnorketamine, memantine, amantadine, phencyclidine (PCP), rolicyclidine (PCPy), PCM, PCPr, tenocyclidine (TCP), tiletamine, 2-oxo-PCP, 2-oxo-PCE, gacyclidine, 3-MeO—PCP, 3-OH—PCP, 4-MeO—PCP, deschloroketamine, 2-fluoro-deschloroketamine, eticyclidine (PCE), 3-MeO—PCE, 4-MeO—PCE, 3-OH—PCE, methoxetamine, methoxmetamine, methoxpropamine, methoxisopropamine, and hydroxetamine.

71. The method of claim 69, wherein component (ii) is selected from the group consisting of:

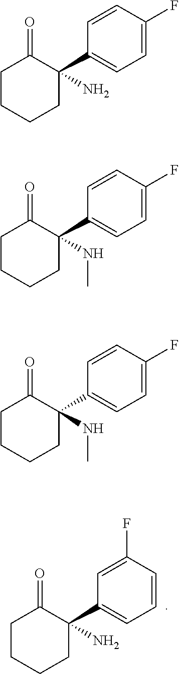 [SMILES: N[C@]1(c2ccc(F)cc2)CCCCC1=O ; CN[C@]1(c2ccc(F)cc2)CCCCC1=O ; CN[C@@]1(c2ccc(F)cc2)CCCCC1=O ; N[C@]1(c2cccc(F)c2)CCCCC1=O]

72. The method of claim 69, wherein component (ii) is selected from the group consisting of ketamine, R-ketamine, S-ketamine, and S-norketamine.

73. The method of claim 69, wherein component (ii) is:

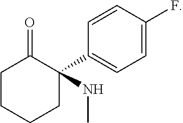 [SMILES: CN[C@@]1(c2ccc(F)cc2)CCCCC1=O]

74. The method of claim 69, wherein component (ii) is selected from the group consisting of ketamine, R-ketamine, and S-ketamine.

75. The method of claim 68, wherein component (ii) is ketamine or an analogue thereof.

76. The method of any one of claims 31-67, wherein component (ii) is selected from the group consisting of 8A-PDHQ, dextromethorphan, dextromethorphan-OD3, dextrorphan, dextrallorphan, dextromethadone (REL-1017), PD-137889, neramexane, memantine, NEFA, dizocilpine, lanicemine, diphenidine, ephenidine, isopropylphenidine, methoxphenidine, fluorolintane, xenon, krypton, nitrous oxide, etoxadrol, dexoxadrol, ibogaine, and noribogaine.

77. The method of claim 76, wherein component (ii) is selected from the group consisting of dextromethorphan and dextromethorphan-OD3.

78. The method of claim 77, further comprising administering to the subject: (iii) a pharmaceutically effective amount of a third compound selected from the group consisting of quinidine and bupropion.

79. The method of any one of claims 31-67, wherein component (ii) is a compound that selectively blocks or negatively modulates NMDA receptors containing the GluN2B (GRIN2B) subunit.

80. The method of claim 79, wherein component (ii) is selected from the group consisting of MIJ821, rislenemdaz, eliprodil, ifenprodil, besonprodil, traxoprodil, radiprodil, BMS-986169, BMS-986163, Ro 25-6981, Ro 04-5595, TCN 237, TCS 46b, and Co 101244.

81. The method of any one of claims 31-67, wherein component (i) and/or component (ii) is in a pharmaceutically acceptable carrier.

82. A pharmaceutical composition comprising the following components:
(i) a positive allosteric modulator (PAM) of the AMPA receptor, or a pharmaceutically acceptable salt thereof; and
(ii) (2R,3S)-2-amino-3-hydroxy-3-pyridin-4-yl-1-pyrrolidin-1-yl-propan-1-one, or a pharmaceutically acceptable salt thereof.

83. The composition of claim 82, wherein component (i) is PF-04958242 (BIIB-104; pesampator).

84. The composition of claim 83, wherein each dose unit of the composition contains between about 0.05 and about 1 mg of PF-04958242 (BIIB-104; pesampator).

85. The composition of claim 84, wherein each dose unit of the composition contains between about 0.1 and about 0.5 mg of PF-04958242 (BIIB-104; pesampator).

86. The composition of claim 82, wherein each dose unit of the composition contains between about 10 and about 100 mg of component (ii).

87. The composition of claim 86, wherein each dose unit of the composition contains between about 20 and about 40 mg of component (ii).

88. The composition of claim 82, further comprising a pharmaceutically acceptable carrier.

89. A method of treating a subject afflicted with cognitive impairment associated with schizophrenia, comprising administering to said subject pharmaceutically effective amounts of the following therapeutic compounds:
(i) a positive allosteric modulator (PAM) of the AMPA receptor, or a pharmaceutically acceptable salt thereof; and
(ii) (2R,3S)-2-amino-3-hydroxy-3-pyridin-4-yl-1-pyrrolidin-1-yl-propan-1-one, or a pharmaceutically acceptable salt thereof.

90. The method of claim 89, wherein therapeutic compounds (i) and (ii) are administered to the subject as a single pharmaceutical composition in which therapeutic compounds (i) and (ii) are present in pharmaceutically effective amounts.
